SEQUENCE LISTING (4 sequences)

SEQ ID NO: 1 - DNA, 24 bp, Artificial Sequence
  Description of Artificial Sequence Synthetic primer
ctgatggtcaagatccagaggtct

SEQ ID NO: 2 - DNA, 25 bp, Artificial Sequence
  Description of Artificial Sequence Synthetic primer
ctgcatgtgcttcatgaaggactct

SEQ ID NO: 3 - DNA, 20 bp, Artificial Sequence
  Description of Artificial Sequence Synthetic primer
tgttaccaactgggacgaca

SEQ ID NO: 4 - DNA, 20 bp, Artificial Sequence
  Description of Artificial Sequence Synthetic primer
ctgggtcatcttttcaccgt

(19) United States
(12) Patent Application Publication
(10) Pub. No.: US 2008/0131432 A1
(43) Pub. Date: Jun. 5, 2008

(54) Method of treating the chemotoxic effects of chemotherapeutic drugs on brain tissue

(75) Inventors: Jitbanjong Tangpong, Lexington, KY (US); Daret St. Clair, Lexington, KY (US); Allan Butterfield, Lexington, KY (US); Mary Vore, Lexington, KY (US); Steven Estus, Lexington, KY (US); William St. Clair, Lexington, KY (US)

Correspondence Address: CROWELL & MORING LLP;INTELLECTUAL PROPERTY GROUP, P.O. BOX 14300, WASHINGTON, DC, 20044-4300, US

(73) Assignee: University of Kentucky Research Foundation, Lexington, KY (US)

(21) Appl. No.: 11/819,735

(22) Filed: Jun. 28, 2007

Related U.S. Application Data
(63) Continuation of application No. 11/592,272, filed on Nov. 3, 2006, now abandoned.
(60) Provisional application No. 60/732,992, filed on Nov. 3, 2005.

(51) Int. Cl. A61K 39/395; A61P 43/00

(52) U.S. Cl. 424/134.1; 424/130.1; 424/133.1; 424/145.1

(57) ABSTRACT

This invention is related to a novel method of treating the chemotoxic effects on the central nervous system of chemotherapeutic drugs, namely Adriamycin, by administering a therapeutically effective amount of anti-Tumor Necrosis Factor-α antibody to a patient to alleviate the symptoms of the effects. The antibody operates to decrease the serum level of Tumor Necrosis Factor-α, prevents the p53 translocation to mitochondria and prevents the decline in mitochondrial respiration of brain tissues and thereby relieves the symptoms of somnolence caused by chemotherapeutic drugs.

### CROSS-REFERENCE TO PRIOR APPLICATIONS

This application claims priority to Provisional Application No. 60/732,992 filed Nov. 3, 2005 having Jitbangong Tangpong as the first named inventor and is incorporated in its entirety by reference herein.

### STATEMENT REGARDING FEDERALLY SPONSORED RESEARCH

This work was supported, in part by NIH grants to DAB [AG-10836, AG-05119] and DSC [AG-05119, CA-80152 and CA-94853].

### FIELD OF THE INVENTION

This invention is related to a novel method of treating the chemotoxic effects on the central nervous system of chemotherapeutic drugs, namely Adriamycin, by administering a therapeutically effective amount of anti-Tumor Necrosis Factor-α antibody to a patient to alleviate the symptoms of the effects. The antibody operates to decrease the serum level of Tumor Necrosis Factor-α, prevents the p53 translocation to mitochondria and prevents the decline in mitochondrial respiration of brain tissues and thereby relieves the symptoms of somnolence caused by chemotherapeutic drugs.

### BACKGROUND OF THE INVENTION

The following includes information that may be useful in understanding the present inventions. It is not an admission that any of the information provided herein is prior art, or material, to the presently described or claimed inventions, or that any publication or document that is specifically or implicitly referenced is prior art.

The present invention relates to, but is not limited to, the fields of toxicology and neurology. The invention relates, for example, to a novel and useful method of preventing or treating the possible chemotoxic side effects of chemotherapeutic drugs on brain tissue, specifically, and the central nervous system generally.

Chemotherapy is the use of medicines or drugs to treat disease, particularly cancer. Chemotherapy can destroy cancer cells that have metastasized or spread to parts of the body far from the primary, or original, tumor because it works throughout the entire body, unlike radiation that targets specific areas. Chemotherapy is a powerful and effective drug for treating cancer, but does have many side effects. The toxic effects of chemotherapy can include, but are not limited to, nausea, vomiting, fatigue, hair loss, sore mouth, skin discoloration, sensitivity to the sun, infertility and diarrea. It is desirable to discover new ways to combat the many side effects of chemotherapeutic drugs, specifically chemotherapeutic drugs that can generate reactive oxygen species (RONS) and/or ionizing radiation.

Adriamycin (hereinafter referred to as ADR), an antibiotic produced by the fungus Streptomyces peuctius, is a potent anticancer drug commonly used in chemotherapy for a variety of cancers, including breast cancer (Hitchcock-Bryan et al., Med. Pediatr. Oncol., 14:211-5 (1986); Fisher et al., J. Clin. Oncol. 7:572-82 (1989)). However, its clinical effectiveness is limited by its toxic effect on normal tissues (Singal et al., J. Mol. Cell. Cardiol. 19:817-28 (1987); Meredith et al., Biochem. Pharmacol. 32:1383-8 (1983); Oteki et al., Biochem. Biophys. Res. Commun. 332(2):326-31 (2005)). These toxic effects include a cumulative, dose-related cardiomyopathy (Singal et al, N. Eng. J. Med. 339:900-5 (1998)). Recent studies in breast cancer survivors have shown persistent changes in cognitive function, including memory loss, tendency for distractions, and difficulty in performing multiple tasks, following chemotherapy using ADR (Schagen et al., Cancer 85:640-50 (1999); Brezden et al., J. Clin. Oncol. 18:2665-70 (2000)).

These studies report that cognitive deficits, particularly in the areas of memory and concentration, are associated with cancer chemotherapy regimens. These defects occur both in the short-term after treatment, as well as up to 2 years and more than 5 years after diagnosis (Ahles et al., J. Clin. Oncol. 20:485-93 (2002); Ferrell et al., Oncology 11:565-76 (1997)). These cognitive problems, collectively called somnolence (or cognitive dysfunction) are also reported in cancer patients undergoing ADR-based chemotherapy. This is especially common in breast cancer patients (Freeman et al., Clin. Breast Cancer 3:S91-9 (2002); Schagen et al., J. Neurooncol. 51(2):159-65 (2001)).

A patient undergoing chemotherapy is experiencing much stress and discomfort. The side effects are particularly disruptive and make a difficult time even worse. It important to minimize a patient's suffering during the course of chemotherapy so they may continue to lead as full of a life as possible. Thus, new agents, compositions and methods for using these agents and compositions which alleviate the toxic effects caused by chemotherapeutic drugs are needed. The present invention provides effective methods to relieve the symptoms of somnolence caused by chemotherapy.

### SUMMARY OF THE INVENTION

The inventions described and claimed herein have many attributes and encompass many embodiments including, but not limited to, those set forth in this Summary. The inventions described and claimed herein are not limited to, or by, the features or embodiments identified in this Summary, which is included for purposes of illustration only and not restriction. The invention provides a new method for treating the toxic effects of chemotherapeutic drugs. The method is preferably directed towards treatment of cognitive defects associated with the use of Adriamycin as a chemotherapy.

Accordingly, one aspect of the invention is to provide a method of treating the toxic effects of chemotherapeutic drugs on the central nervous system comprising administering anti-Tumor Necrosis Factor-α antibody to a patient in a therapeutically effective amount to alleviate the symptoms. In a further aspect of the invention the chemotherapeutic drug is Adriamycin.

Another aspect of the invention is to demonstrate a method of decreasing the serum level of Tumor Necrosis Factor-α comprising administering anti-Tumor Necrosis Factor-α antibody to a patient in a therapeutically effective amount to alleviate the toxic effects of chemotherapeutic drugs on the central nervous system. In a further aspect of the invention the chemotherapeutic drug is Adriamycin.

Yet another aspect of the invention is to provide a method of preventing the decline in mitochondrial respiration of brain tissues caused by the use of chemotherapeutic drugs comprising administering anti-Tumor Necrosis Factor-α antibody to a patient in a therapeutically effective amount to alleviate the toxic effects of chemotherapeutic drugs on the central nervous system. In a further aspect the chemotherapeutic drug is Adriamycin.

In another aspect of the invention a method is demonstrated that prevents p53 translocation to mitochondria comprising administering anti-Tumor Necrosis Factor-α antibody to a patient in a therapeutically effective amount to alleviate the toxic effects of chemotherapeutic drugs on the central nervous system. In a further aspect the chemotherapeutic drug is Adriamycin.

In still another aspect of the invention a method is presented that provides for alleviating the symptoms of somnolence comprising administering anti-Tumor Necrosis Factor-α antibody to a patient in a therapeutically effective amount to alleviate the toxic effects of chemotherapeutic drugs on the central nervous system. In a further aspect the chemotherapeutic drug is Adriamycin.

In all of the above methods it is another aspect of the invention that the anti-Tumor Necrosis Factor-α antibody is etanercept. In yet another aspect the anti-Tumor Necrosis Factor-α antibody is infliximab. In another aspect the anti-Tumor Necrosis Factor-α antibody is adalimumab. In still another aspect the anti-Tumor Necrosis Factor-α antibody is any humanized monoclonal antibody specific for Tumor Necrosis Factor-α. In another aspect of the invention the anti-Tumor Necrosis Factor-α antibody is any molecule that contains extracellular portions of a human Tumor Necrosis Factor-α receptor.

In another aspect the use of the anti-Tumor Necrosis Factor-α antibody to block TNF can prevent secondary cancers after cancer treatment.

### BRIEF DESCRIPTION OF THE FIGURES

FIG. 1: Shows Adriamycin localization (orange-red fluorescence) in mouse brain 3 hours after 20 mg/kg ADR, was apparent in the choroid plexus. (A), ADR alone; (B), anti-TNF α antibody immediately followed by ADR; (C), Saline; (D), anti-TNF α antibody alone. The ADR orange-red fluorescence was not detected in cortex (E), and hippocampus (F). Magnification 20×.

FIG. 2: Shows Adriamycin increased circulating TNF α. TNF α levels are significantly elevated in mice 3 hours after 20 mg/kg ADR compared with saline control (*p<0.001). The levels of TNF α were increased at the earliest time point examined.

FIG. 3: Shows that Adriamycin-induced TNF α is increased in brain tissues. TNF α levels were significantly increased in mice 3 hours after treatment with a single dose of 20 mg/kg ADR compared with the saline control (*p<0.01). The levels of TNF α were not different in mice treated with anti-TNF α antibody (TNF α-Ab), immediately followed by ADR. Anti-TNF α antibody treatment alone did not increase TNF α levels in the brain.

FIG. 4: Shows the immunofluorescence analysis of TNF α localization in cortex following ADR treatment. Confocal microscopy analysis of TNF α (red) and neurons (MAP2, green) and colocalization (yellow) showed that TNF α is increased in neurons of mice 3 hours after treatment with 20 mg/kg ADR compared to mice treated with saline (A), anti-TNF-α antibody together with ADR or anti-TNF α antibody alone (B). Pictures are representative images from at least 3 independent experiments per group.

FIG. 5: Demonstrates the immunofluorescence analysis of TNF α localization in hippocampus following ADR treatment. Confocal microscopy analysis of TNF α (red) and neurons (MAP2, green) and colocalization (yellow) showed that TNF α is increased in neurons of mice 3 hours after treatment with 20 mg/kg ADR compared to mice treated with saline (A), anti-TNF α-antibody together with ADR or anti-TNF α antibody alone (B). Pictures are representative images from at least 3 independent experiments per group.

FIG. 6: Shows that Adriamycin-mediated TNF α elevation leads to mitochondrial dysfunction. Brain mitochondrial respiration was determined using pyruvate+malate as substrates in mitochondria from mice treated with saline or ADR. The results show mitochondrial respiration complex I, pyruvate+malate as substrates, was significantly decreased (* p<0.05 compared to all other groups) 3 hours after treatment with ADR (20 mg/kg), and the mitochondrial respiration decline was blocked by anti-TNF α antibody. Data represent the mean±SEM of six independent experiments.

FIG. 7: Shows representative immunoblots showing the levels of p53, Bax, Bcl-xS, Bcl-xL, and MnSOD in mitochondria. Mitochondrial proteins were isolated from brain tissues of ADR- and saline-treated mice, and separated by SDS-polyacrylamide gel electrophoresis. The pro-apoptotic proteins, p53, was significantly increased at 3, 6, and 24 hours (*P<0.01), and the pro-apoptotic protein, Bax was increased at 3 and 6 hours (*P<0.05). The anti-apoptotic protein, Bcl-xL show an increase in protein density 6, 24, 48, 72 hours after treatment with ADR (*P<0.05). Succinate dehydrogenase is used to normalize protein loading. The results shown are a representative independent set of data of n=3 separate sets from individual animals.

FIG. 8: Shows representative immunoblots showing the prevention of p53 and Bax translocation to mitochondria by anti-TNF α antibody. Western blot analyses of p53, Bax, Bcl-xS, Bcl-xL, and succinate dehydrogenase demonstrate that in mitochondrial proteins isolated from brain tissues 3 hours after mice were treated with ADR, IgG followed by ADR, and anti-TNF α antibody, IgG, or saline as a control. Only the pro-apoptotic proteins, p53 and Bax show an increase in protein density after treatment with ADR or IgG followed by ADR (*P<0.05), while no differences in Bcl-xL are observed at the 3 hour period following ADR treatment. Anti-TNF treatment immediately prior blockedp53 and BAX translocation to mitochondria following ADR treatment. Suxxinate dehydrogenase was used to normalize for protein loading. These results shown are a representative set of data of n=3 separate sets from individual animals.

FIG. 9: Shows representative co-immunoprecipitation of the pro-apoptotic protein, p53, in mitochondria with anti-apoptotic protein, Bcl-xL. (A), Brain mitochondria were isolated from mice after ADR and saline control treatment, and were immunoprecipitated with anti-p53 antibody. Bands specific for Bcl-xL were increased at 3 hours (*P<0.05). (B) Blocking with anti-TNF α antibody prevented p53 translocation to mitochondria and co-localization with Bcl-xL in mice 3 hours after injection with anti-TNF α antibody immediately followed by ADR compared to mice treated with ADR or IgG followed by ADR (*P<0.05). Similar immunoprecipitation studies employing IgG using preimmune serum reveal no band of Bcl-xL in the mitochondrial pellet fraction. The results shown are a representative set of data of n=3 separate sets from individual animals.

FIG. 10: Shows ADR-induced cytochrome c release from mitochondria to cytosol. Western blot analyses of cytochrome c and succinate dehydrogenase or β-actin were determined in mitochondrial or cytosolic fractions isolated from brain tissues 3 hours after mice were treated with ADR, and anti-TNF antibody immediately followed by ADR, and anti-TNF antibody, IgG, or saline as controls. Cytochrome c was decreased in mitochondria (*P<0.01) but increased cytosol after treatment with ADR or IgG followed by ADR. Succinate dehydrogenase or β-actin were used to normalize for protein loading. The Western blots shown a re a representative set of data of n=3 separate sets from individual animals. Quantification of all the data

FIG. 11: Shows ADR-induced caspase 3 activity in brain tissues. Caspase 3 activity is significantly increased in mice 3 and 72 hours following ADR treatment compared with saline control (**P<0.001) and also increased at 72 hours compared with 3 hours after 20 mg/kg ADR (**P<0.05). These results shown are a representative set of data of n=3 separate sets from individual animals.

FIG. 12: Shows ADR-induced TUNEL-positive apoptotic cell death in cortical and hippocampal regions of the brain. (A) Brain cryosection tissues were treated with biotinylated rTdT, followed by streptavidin peroxidase and diaminobenzidine-hydrogen peroxide, and counterstained with methyl-green. The nuclei are represented by the positive dark brown staining. (B) Following 3 and 72 hours after ADR treatment, an increased number of apoptotic cells were found in cortical and hippocampal cells compared with saline (*P<0.05). n=3 separate animals were studied for each group and time point. (For interpretation of the references to colour in this figure legend, the reader is referred to the web version of this article.)

FIG. 13: Shows adriamycin increased circulating TNF-α. (a) TNF-α levels are significantly elevated in both wild-type and iNOSKO mice after 20 mg/kg ADR for 3 hours compared with saline control (*p<0.001). (b, c) Exogenous injected TNF-α increased the circulating TNF-α level (*p<0.001). (b) Neutralizing antibody against circulating TNF-α abolished increased TNF-α in serum 3 hours after treatment with ADR. (c) DPTA NONOate had no effect on circulating TNF-α levels. Preimmune IgG injection was used as a negative control.

FIG. 14: Adriamycin-induced iNOS MRNA expression in wild-type mice. Representative RT-PCR product of ADR-induced iNOS MRNA in brain tissues after treatment with ADR, TNF-α or LPS for 3 hours compared with saline control. Blocking circulating TNF-α with anti-TNF-α antibody prevented ADR-mediated iNOS increase in the brain tissues.

FIG. 15: Shows adriamycin-mediated TNF-α elevation leads to mitochondrial dysfunction. Brain mitochondrial respiration was determined using pyruvate+malate as substrates. (a) Mitochondrial respiration complex I, pyruvate+malate as substrates, was significantly decreased in wild-type mice 3 hours after treatment with ADR (20 mg/kg) compared with saline control (*p<0.05), but not in iNOSKO mice. (b) The mitochondrial respiration decline was blocked by anti-TNF-α antibody. Exogenous TNF-α treatment for 3 hours led to mitochondrial respiration complex I being significantly decreased compared with saline or IgG treatment groups in wild-type mice (*p 0.05), but not in iNOSKO mice. (c) DPTA NONOate causes mitochondrial dysfunction in iNOSKO mice compared with the same genotype mice treated with saline, IgG, ADR or TNF-α (*p<0.05). Data represent the mean±SEM of three independent experiments.

FIG. 16: Shows ADR-induced protein nitration. (a) Immunoreactive levels of nitrotyrosine-adducted proteins from isolated brain mitochondria of mice 3 hours after treatment with ADR 20 mg/kg or saline control. The results show significantly increased protein nitration in ADR-treated wild-type mice, but no difference with controls in iNOSKO mice (*p<0.01). (b, c) Immunoprecipitration coupled to western blot analyses showed the level of nitrotyrosine in MnSOD significantly increased 3 hours after 20 mg/kg ADR treatment in wild-type mice, but not in iNOSKO mice similarly treated (*p<0.01). Mitochondria, isolated from brain, treated with 20 μM peroxynitrite, was used as a positive control and IgG was used as negative control. (b) Representative western blots of the nitrated MnSOD. (c) The quantitative analysis of nitrated MnSOD protein density. Results are reported as mean±SEM of nitrotyrosine-adducted MnSOD protein density from three independent experiments.

FIG. 17: Shows ADR-induced MnSOD inactivation. Brain homogenates were used for SOD activity assay. (a) MnSOD activity was significantly decreased in wild-mice after treatment with ADR 20 mg/kg for 3 hours (*p<0.05). The MnSOD was not changed in iNOSKO mice similarly treated. (b) L-NAME, a NOS inhibitor, led to increased MnSOD activity compared with that from saline- or ADR-treated mice (*p<0.001). L-NAME prevented MnSOD inactivation but increased enzymatic activity after treatment with ADR in wild-type mice (***p<0.001). Data represent the mean+SEM of three independent experiments.

FIG. 18: Shows no change in MnSOD and CuZnSOD protein levels in ADR-treated mice. Representative western blots of brain homogenate proteins from mice 3 hours after treatment with ADR 20 mg/kg or saline. Immunodetection with the polyclonal anti-MnSOD and anti-Cu/ZnSOD antibody exhibited no changed in protein density levels of MnSOD or CuZnSOD in both wild-type mice and iNOSKO mice after treatment with ADR or saline. Data represent the mean±SEM of three independent experiments. Top: representative western blot of MnSOD, CuZnSOD and G-3-PDH, the latter used for normalization of protein loading. Bottom: representative protein density of MnSOD and CuZnSOD after normalization with G-3-PDH.

FIG. 19: Shows MnSOD protein nitration was quantified by immunoprecipitation using anti-MnSOD antibody, and probed with anti-3-nitrotyrosine antibody as described in Methods. (a) No significant difference was observed in the expression of proteins between control and ADR-treated brain mitochondria. (c) A significantly increased protein nitration was observed in ADR-treated mitochondrial sample compared with that of control (p<0.04). (b) Ponceaustained blot. (d) and (e) Histograms from (a) and (c), respectively.

FIG. 20: Shows MnSOD protein was immunoprecipitated using anti-MnSOD antibody as described in Methods. Supernatant fluids were used to carry out 2D gel electrophoresis before (a) and after (d) immunoprecipitation, which showed a missing spot corresponding to MnSOD (indicated by box).

### DETAILED DESCRIPTION OF THE INVENTION

### Definitions and Acronyms

In accordance with the detailed description, the following abbreviations and definitions apply. It must be noted that as used herein, the singular forms “a”, “and”, and “the” include plural referents unless the context clearly dictates otherwise. Thus, for example, reference to “an antibody” includes a plurality of such antibodies and reference to “the dosage” includes reference to one or more dosages and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Definitions

By the term “subject” or “patient” as used herein is meant to include a mammal. The mammal can be a canine, feline, primate, bovine, porcine, camelide, caprine, rodent, or equine. Preferably, the mammal is human.

By “Anti-Tumor Necrosis Factor α” is meant any anti-tumor necrosis factor a drug used for the treatment of inflammatory related diseases including, but not limited to etanercept, adalimumab and infliximab.

The term “efficacy” as used herein refers to the effectiveness of a particular treatment method. Efficacy can be measured based on such characteristics, but not limited to, the relief of symptoms, the alleviation of symptoms, the lessening of symptoms, the reduction of symptoms, the cure of symptoms, and the like.

The term “to alleviate the symptoms” as used herein is intended to mean a lessening or reduction of the symptoms of somnolence, including but not limited to a reduction in, but not limited to, the cognitive defects associated with the use of chemotherapeutic drugs, including but not limited to Adriamycin.

The term “chemotherapeutic drugs” as used herein is intended to refer to any drugs, agents or compositions that are used in the treatment of cancer including but not limited to Adrimycin.

The terms “treating” and “treatment” and the like are used herein to generally mean obtaining a desired physiological effect. More specifically, the methods described herein which are used to treat a subject suffering from the toxic effects of chemotherapeutic drugs are provided to effectively relieve any, but not limited to, one of the following: central nervous system damage, somnolence, cognitive dysfunction, memory loss, concentration loss, lethargy, loss of appetite, irritability, and drowsiness.

By “therapeutically effective amount” is meant an amount of an agent, reagent, compound, composition or combination of such disclosed herein that when administered to a mammal is sufficient to relieve the toxic effects of chemotherapeutic drugs.

Acronyms

The following acronyms are commonly used for the associated terms and would be known in the art:

ADR Adriamycin

anti-TNF α Anti-Tumor Necrosis Factor α

CNS central nervous system

G-3-PDH glyceraldehyde-3-phosphate dehydrogenase

hours hour

HEPES 4-(2hydroxyethyl)-1-piperrazineethanesulfonic acid

i.p. intraperitoneal

iNOSKO inducible form of nitric oxide

MAP2 microtubule-associated proteins

MnSOD manganese superoxide dismutase

PBS phosphate buffered solution

RCR respiration Control ratios

RONS reactive oxygen and nitrogen species

RONS reactive oxygen/nitrogen species

TBST Tris-buffered saline and Tween 20

TNF α Tumor Necrosis Factor α

In this invention and the following examples, it is demonstrated that ADR accumulates only in areas outside the blood brain barrier, but increased TNF α levels are found in serum and both the hippocampal and cortical regions of the brain. Mitochondrial function is altered in the brain following ADR treatment. Furthermore, a neutralizing antibody against TNF α given systemically abolishes the observed TNF α levels in brain tissue. The results support the hypothesis that TNF α is an important mediator of the observed ADR-induced mitochondrial dysfunction.

The Toxic Effects of Chemotherapeutic Drugs

Systemic TNF α is well recognized as a signal in the complex network of immune-neuron interaction, affecting the CNS (Besedovsky et al., Endocr. Rev. 17:64-102 (1996); Blattleis et al., Ann. NY Acad. Sci. 840:608-18 (1998) Licinio et al., J. Clin. Invest. 100:2941-7 (1997)). This effect is seen in diseases such as HIV and Alzheimer's (Eric et al., JIADS 31:S43-54 (2002); Mattson et al., Cell Death and Differentiation 1350-9047/05:1-12 (2005); Valcour et al., AIDS 8:79-86 (2004); Greig et al., Ann NY Acad Sci. 1035:290-315 (2004)). The finding that tissue levels of TNF α increase in brain tissue is consistent with the effect of ADR on circulating TNF α levels. Circulating TNF α may enter the brain by transport across the blood brain barrier and cause stimulation of local TNF α production after physically entering the brain (Gutierrez et al., J. Neuroimmuno.l 47:169-76 (1993); Osburg et al., Am. J Physiol. Endocrinol. Metab. 283:E899-908 (2002)).

Alternatively, increased brain TNF α levels may result from the activation of microglia and macrophages that enter the brain after ADR treatment. TNF α may act on brain cells to cause the observed decline in mitochondrial respiration and subsequent increase in oxidative stress markers. The successful alleviation of the decline in mitochondrial respiration of brain tissue supports the concept that the neutralizing antibody acts to reverse the effects of the administration of ADR.

Cancer patients undergoing chemotherapy often suffer from a somnolence syndrome (also known as cognitive dysfunction), also called “chemobrain”. Although the biochemical basis for these cognitive problems is unknown, it has been demonstrated that cancer therapeutic agents such as ADR can modulate endogenous levels of cytokines such as tumor necrosis factor alpha (TNF α) (Usta et al., IPNA 4:1538-5 (2004)).

Enhanced circulating TNF α can initiate local TNF α production via activation of glia cells leading to production of reactive oxygen/nitrogen species (RONS) (Szelenyi, Brain Research Bulletin 54:329-38 (2001)). RONS, including superoxide, hydrogen peroxide, and nitric oxide, can react directly with each other or indirectly to generate even more reactive species (Halliwell and Gutteridge, Free Radicals in Biology and Medicine, 3rd Ed., pp. 246-343 (1999)). It was recently reported that seventy-two hours after a single intraperitoneal injection of ADR, there was a significant increase in levels of protein oxidation and lipid peroxidation in brain tissues (Joshi et al., Free Radic. Res. in press (2005)). However, the mechanism by which ADR causes oxidative stress in the brain previously was unknown.

Treating the Toxic Effects of Chemotherapeutic Drugs

In one aspect, the method disclosed herein can be used to treat the toxic effects of chemotherapeutic drugs on the central nervous system by the administration of an anti-Tumor Necrosis Factor α antibody to a patient. The effects or symptoms treated include, but are not limited to, central nervous system damage, somnolence, cognitive dysfunction, memory loss, concentration loss, lethargy, loss of appetite, irritability, and drowsiness. In another aspect of the invention the chemotherapeutic drug is Adriamycin, although the invention is useful for any chemotherapeutic drug that generate RONS and/or ionizing radiation.

It is well established that ADR does not cross the blood brain barrier (Bigotte et al., Acta. Neuropathol. 57:121-9 (1982); Bigotte et al., Acta. Neuropathol. 58:193-202 (1982)) However, circulating levels of TNF α can directly pass the blood brain barrier and activate microglia and neurons to further increase local TNF α levels (Osburg et al., Am. J Physiol. Endocrinol. Metab. 283:E899-908 (2002)). TNF α is known to induce neuronal damage (Gutierrez et al., J. Neuroimmunol. 47:169-76 (1993)).

By administering anti-TNF α antibody in a therapeutically effective amount the level of TNF α is decreased, thereby alleviating or eliminating the side effects of the chemotherapeutic drugs. The anti-TNF α antibody can be used alone or combination with other therapies, drugs, compositions, agent or reagents. Furthermore, the administration of the anti-TNF α antibody can be used to alleviate the symptoms of somnolence in patients undergoing chemotherapy.

The amount or concentration of the anti-TNF α antibody administered that is necessary to achieve the therapeutic effects may be dependent on an individual patient's level of TNF α. generated in each patient after treatment with a chemotherapeutic agent. As the concentration or level of the TNF α generated in a patient by the chemotherapeutic drug increases the amount of anti-TNF α antibody necessary to neutralize the TNF α. and achieve the desired therapeutic effect will also increase.

Decreasing the Serum Level of TNF α

One aspect of the invention provides a method of decreasing the serum level of Tumor Necrosis Factor-α in patients being treated with chemotherapeutic drugs. The chemotherapeutic drug may be, but is not limited to, Adriamycin. As is demonstrated in Example 2, TNF α levels are significantly higher when a subject is treated with ADR. By administering anti-Tumor Necrosis Factor-α antibody to a patient in a therapeutically effective amount the toxic effects of chemotherapeutic drugs on the central nervous system can be alleviated with the decrease of serum levels of TNF α.

The amount or concentration of the anti-TNF α antibody administered that is necessary to achieve the therapeutic effects may be dependent on an individual patient's level of TNF α. generated in each patient after treatment with a chemotherapeutic agent. As the concentration or level of the TNF α generated in a patient by the chemotherapeutic drug increases the amount of anti-TNF α antibody necessary to neutralize the TNF α. and achieve the desired therapeutic effect will also increase.

Preventing the Decline of Mitochondrial Respiration in Brain Tissue

Another aspect of this invention prevents the decline of mitochondrial respiration in brain tissue, so as to alleviate the side effects on the CNS associated with chemotherapeutic drugs. TNF α-induced tissue injury is mediated, at least in part, by its effect on mitochondria (Goossens et al., Proc. Natl. Acad. Sci. USA 92:8115-9 (1995)). TNF α induces morphologic damage of mitochondria and biochemical respiratory defects in cultured cells (Liu et al., J. of Immunolo. 172:1907-15 (2004); Schulze-Osthoffet al., J. Biol. Chem. 267:5317-23 (1992)). The cytotoxicity of TNF α depends on the induction of the mitochondrial permeability transition pore (Lancaster et al., FEBS Lett 248:169-74 (1989)). Thus, an increase in TNF α levels can be a link between ADR-induced oxidative stress and CNS injury.

The present invention and examples demonstrate that ADR autofluorescence is detected only in areas of the brain located outside the blood-brain barrier, but that a strong tumor necrosis factor alpha (TNF α) immunoreactivity is detected in the cortex and hippocampus of ADR-treated mice. Systemic injection of ADR leads to a decline in brain mitochondrial respiration via complex I substrate shortly after ADR treatment (p<0.05). The levels of the known anti-apoptotic protein, Bcl-xL, and the pro-apoptotic proteins, Bax, are increased in mitochondria isolated from ADR-treated brain tissues. Furthermore, p53 migrates to mitochondria and interacts with Bcl-xL, supporting the hypothesis that mitochondria are targets of ADR-induced CNS injury.

Neutralizing antibodies against circulating TNF α completely abolish both the increased TNF α in the brain and the observed mitochondrial injury in brain tissues. These results are consistent with the notion that TNF α is an important mediator by which ADR induces central nervous system (CNS) injury. This invention and these experiments, the first to provide direct biochemical evidence of ADR toxicity to the brain, revealed novel mechanisms of ADR-induced CNS injury, and offers a therapeutic intervention against circulating TNF α-induced CNS effects.

The amount or concentration of the anti-TNF α antibody administered that is necessary to achieve the therapeutic effects may be dependent on an individual patient's level of TNF α. generated in each patient after treatment with a chemotherapeutic agent. As the concentration or level of the TNF α generated in a patient by the chemotherapeutic drug increases the amount of anti-TNF α antibody necessary to neutralize the TNF α. and achieve the desired therapeutic effect will also increase.

Adriamycin-Mediated Nitration of Manganese Superoxide Dismutase

Nitric oxide (NO) plays a role in ADR-mediated CNS injury. There is a link between cytokine production and inducible nitric oxide synthase (iNOS) in brain tissues. Nitric oxide is synthesized from L-arginine through two distinct enzyme catalyzed pathways. The first pathway is a tightly regulated, constitutively-expressed nitric-oxide synthase found mainly in endothelial and brain cells (Vasquez-Vivar et al. 1999; Kalivendi et al. 2001). The second pathway is a calcium-independent, cytokine-inducible form of nitric oxide synthase (iNOS) (Zeng et al. 2005). This enzyme, once expressed stays active as long as substrates are available.

Within the central nervous system microglia and astrocytes can generate nitric oxide (NO) radicals from iNPS activation (Marcus et al. 2003); Candolfi et al. 2004). NO reacts rapidly with superoxide radicals, thereby limiting the NO lifetime. NO reacts with superoxide radicals to form peroxynitrite (ONOO—), a potent biological oxidant that has been implicated in diverse forms of free radical-induced tissue injury (Ste-Marie et al. 2001; Choi et al. 2002; Rubbo et al. 2002). Inhibition of mitochondrial electron transport and inactivation of FeS-containing enzymes by NO have been demonstrated (MacMillan-Crow et al. 1996; Radi 2004). Thus it is possible that increased production of NO may participate in the development of neurotoxicity by generating peroxynitrite and inactivating some key components of the mitochondrial defense system.

ADR treatment led to an increase in NO in breast cancer cells in vitro, and is reportedly cytotoxic to cells in vivo (Ozen et al. 2001; Guorffy et al. 2005). The mechanism by which NO production is stimulated by these agents is not clear. It has been shown that nuclear factor-kappa B (NF-kB) enhancer elements regulate cytokine-mediated induction of the inducible NOS gene (Kin et al. 1999; Akama and Van Eldik 2000). NF-kB is a redox-sensitive transcription factor that has been shown to be activated by oxidizing agents, such as hydrogen peroxide (Schreck et al. 1992; Santoro et al. 2003: Snyder and Morgan 2005). Therefore, it is possible that increased NO production by ADR is a result of TNF-α-mediated NF-kB activation of inducible nitric oxide synthase (iNOS) expression. As both NO and superoxide radicals can be generated in mitochondria it is likely that both radicals and the resulting reactive species are, in part, responsible for normal tissue injury during cancer therapy.

An important primary antioxident defense in the mitochondria is the manganese superoxide dismutase (MnSOD). Inactivation of the MnSOD gene in mammals yielded detrimental results (Ii et al. 1998).Thus lack of MnSOD activity can severely impair mitochondria and effect the brain, both of which have high demands for oxidative metabolism. Overexpression of MnSOD protects against numerous agents and conditions that cause oxidative stress and/or neuronal injury (Gonzalez-Zulueta et al. 1998; Keller et al. 1998; Li et al. 1998). Therefore, a high-level of MnSOD activity is needed for protection of neuronal cells in conditions where overproduction of ROS or RNS is involved. Although Applicants do not wish to be bound by any particular theory, mitochondrial alteration demonstrates that NO could be involved in the elevated oxidative stress in the brain following ADR treatment and suggests a link between cytokine production and iNOS induction in brain tissues.

Preventing p53 Translocation to Mitochondria

The results disclosed in the following examples indicate that ADR induced a decline in brain mitochondrial respiration complex I, but had no effect on complex II. This is consistent with the possibility that the [4Fe—4S] cluster protein in complex I is inactivated by ROS generated in mitochondria, since the [4Fe—4S] protein of complex I extends into the inner membrane where superoxide is generated. This possibility is strongly supported by previous studies, which demonstrated that transgenic mice overexpressing MNSOD are protected from ADR-induced complex I inactivation in cardiac tissues (Yen et al., J. Clin. Invest. 98:1253-60 (1996); Yen et al. Ach. of Biochem. Biophys. 362:59-66 (1999)). Mitochondrial dysfunction may be one of the signals initiating the mitochondrial apoptosis pathway by translocation of pro-apoptotic proteins, p53 and Bax to mitochondria. The increase of these pro-apoptotic proteins coincides with the increase in mitochondrial dysfunction, suggesting mitochondrial dependent tissue injury.

The finding that p53 interacted with Bcl-xL in mitochondria further supports the role of mitochondria in ADR-induced CNS injury. This data is consistent with recent reports demonstrating that Bax is required for p53 translocation to mitochondria (Chipuk et al., Cancer Cell 4:371 (2003); Chipuk et al., Science 303:1010-4 (2004)). It has been demonstrated that p53 can directly induce permeabilization of the outer mitochondrial membrane by forming complexes with the pro-survival, Bcl-xL protein (Marchenko et al., J. Biol. Chem. 275:16202-12 (2000); Mihara et al., Molecular Cell 11:577-90 (2003); Schuler et al., Biochem. Soc. Trans. 29:684-8 (2001)). Although Applicants do not wish to be bound by any particular theory, the mitochondrial alteration reported in the current study could be involved in the elevated oxidative stress in the brain following ADR treatment (Joshi et al., Free Radic. Res. (2005)). Thus, one aspect of the invention is to provide a method that prevents p53 translocation to mitochondria to alleviate the toxic effects of chemotherapeutic drugs on the central nervous system. In another aspect the chemotherapeutic drug is Adriamycin.

The amount or concentration of the anti-TNF α antibody administered that is necessary to achieve the therapeutic effects may be dependent on an individual patient's level of TNF α. generated in each patient after treatment with a chemotherapeutic agent. As the concentration or level of the TNF α generated in a patient by the chemotherapeutic drug increases the amount of anti-TNF α antibody necessary to neutralize the TNF α and achieve the desired therapeutic effect will also increase.

Administration

Anti-Tumor Necrosis Factor-α antibody may be administered in a variety of ways including, but not limited to, parenteral administration, including subcutaneous (s.c.), subdural, intravenous (i.v.), intramuscular (i.m.), intrathecal, intraperitoneal (i.p.), intracerebral, intraarterial, or intralesional routes of administration, localized (e.g., surgical application or surgical suppository).

Preferably, anti-Tumor Necrosis Factor-α antibody is formulated for parenteral administration in a suitable inert carrier, such as a sterile physiological saline solution. For example, the concentration of anti-Tumor Necrosis Factor-α antibody in the carrier solution is typically, although not limited to, between about 1-100 mg/mL. The dose administered will be determined by route of administration. The dose will also depend on the patient and the amount of TNF α generated or circulating in the patient. Preferred routes of administration include parenteral, subcutaneous, or intravenous administration.

For parenteral administration, anti-Tumor Necrosis Factor-α antibody can be administered as injectable dosages of a solution or suspension of the substance in a physiologically acceptable diluent with a pharmaceutical carrier, which can be a sterile liquid such as water and oils with or without the addition of a surfactant. Other acceptable diluents include oils of animal, vegetable, or synthetic origin, for example, peanut oil, soybean oil, and mineral oil. In general, glycols such as propylene glycol or polyethylene glycol (PEG) are preferred liquid carriers, particularly for injectable solutions. The anti-Tumor Necrosis Factor-α antibody of this invention can be administered in the form of a depot injection or implant preparation, which can be formulated in such a manner as to permit a sustained release of the active ingredient(s). A preferred composition comprises anti-Tumor Necrosis Factor-α antibody, formulation in a buffer.

Therapeutic anti-Tumor Necrosis Factor-α antibody compositions generally are placed into a container having a sterile access port, for example, an intravenous solution bag or vial having a stopper pierceable by a hypodermic injection needle or similar sharp instrument.

Modes of administration include oral, parenteral (e.g., subcutaneous, subdural, intravenous, intramuscular, intrathecal, intraperitoneal, intracerebral, intraarterial, or intralesional routes of administration), topical, localized (e.g., surgical application or surgical suppository), rectal, and pulmonary (e.g., aerosols, inhalation, or powder). Preferably, the route of administration is parenteral. More preferably, the route of administration is intravenous.

The active compound is effective over a wide dosage range and is generally administered in a pharmaceutically or therapeutically effective amount. The therapeutic dosage of the compounds of the present invention will vary according to, for example, the particular use for which the treatment is made, the manner of administration of the compound, the health and condition of the patient, and the judgment of the prescribing physician. For example, for intravenous administration, the dose will typically be in the range of about 0.5 mg to about 100 mg per kilogram body weight, preferably about 3 mg to about 50 mg per kilogram body weight. The effective dose will increase depending on the amount or concentration of TNF α in a particular patient. Effective doses can be extrapolated from dose-response curves derived from in vitro or animal model test systems. Typically, the clinician will administer the compound until a dosage is reached that achieves the desired effect.

An intravenous formulation may be prepared as follows:

| Ingredient | Quantity | |
|---|---|---|
| Active Ingredient | 250.0 | mg |
| Isotonic saline | 1000 | ml |

Therapeutic compound compositions generally are placed into a container having a sterile access port, for example, an intravenous solution bag or vial having a stopper pierceable by a hypodermic injection needle or similar sharp instrument.

Combination Therapy

The methods described by the present invention can be used alone, or in combination with many different therapies, compounds, medications, methods and treatments used to treat the side effects of chemotherapeutic drugs. Different medications that can be used in combination with the present invention include, but are not limited to, Bax solution, Benadryl®, Nystatin, Viscous lidocaine solution, Diflucan®, Compazine®, Decadron®, Zofran®, Kytril®, Lomotil®, Marinol®, Megace®, Reglan®, Imodium®, Milk of Magnesia®, Senokot®, and magnesium citrate.

There are many different treatments and drugs to alleviate the adverse side effects of chemotherapeutic drugs and chemotherapy. Because each patient may suffer from a different type or combination of side effects the treatments used will vary depending on the patient's condition. Therefore a heath care practitioner will have to determine the best treatments to help an individual patient, and will often combine a variety of treatment modalities. Treatment modalities include, but are not limited to drugs, hormones, biology therapy, nutrition or diet regimens and surgery. Any of the methods disclosed herein may be used with any other treatment to alleviate side effects of chemotherapy.

In summary, the results demonstrate that ADR-induced circulating TNF α is causally related to the observed CNS injury. TNF α-induced mitochondrial dysfunction with its downstream consequence leading to further increases in oxidative stress in the brain may, at least in part, be responsible for the cognitive dysfunction (somnolence syndrome) observed in many patients undergoing ADR-based chemotherapy. Although Applicants do not wish to be bound by theory, it is believed that ADR-induced circulating TNF α may be directly responsible for the observed brain mitochondrial dysfunction, or that circulating TNF α may further increase TNF α from activated glia cells.

The results shown in the Examples, supra, demonstrate that a neutralizing antibody against TNF α abolished the observed mitochondrial injury in animals treated with ADR. Antibodies against TNF α have been in clinical use for many inflammatory associated diseases. The present invention is a method of using an antibody to TNF α to alleviate the chemotoxic effects of chemotherapeutic drugs.

### EXAMPLES

The examples below are only representative of some aspects of the invention. It will be understood by those skilled in the art that the invention as set forth in the specification can be practiced in a variety of different ways.

### Example 1

Eight-week-old male B6C3 mice (25-30 g) were kept under standard conditions. The mice were injected in a single intraperitoneal (i.p.) dose of 20 mg/kg adriamycin (Doxorubicin hydrochloride, Gensia Sicor Phamaceuticals, Inc., Irvine, Calif.), or the same volume of saline as control for 3 hours. This dose and time were based on previous studies in that demonstrated ADR induced cardiomyopathy (Yen et al., J. Clin. Invest. 98:1253-60 (1996); Yen et al. Ach. of Biochem. Biophys. 362:59-66 (1999)).

To determine whether the neutralization of TNF α in the periphery would mitigate the brain biochemical effects of ADR, anti-mouse TNF α antibody (R&D Systems, Minneapolis, Minn.) was diluted in saline and immediately injected in a single i.p. dose of 40 ng/kg, and anti-TNF α antibody immediately followed by ADR also was injected to separate animals. Controls consisted of anti-TNF α antibody alone, or saline in the same total volume. All results were obtained from at least three separate experiments.

### Example 1a

### Localization of ADR in Brain Tissue

Example 1a describes the use of an inverted fluoresence microscope to exhibit the accumulation of ADR in brain tissue.

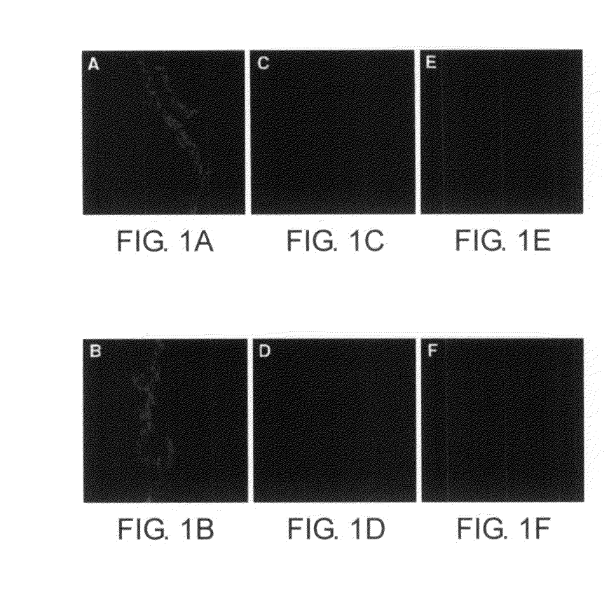

Mice were euthanized by the i.p. injection of 65 mg/kg of nembutal (Sodium pentobarbital, Abbott Laboratories, North Chicago, Ill.), and were perfused via cardiac puncture initially with 0.1 M phosphate buffered saline (PBS), pH 7.4, and subsequent fixation with 4% paraformaldehyde. Brain tissues were removed and coronal cryosection at regular intervals of 7 μm thickness was performed. The sections were prepared for immunohistochemistry and detection of ADR. ADR in brain tissue slices was directly visualized using an inverted fluorescence microscope, FIG. 1 (excitation filter 550 nm, and barrier filter 590 nm). Photomicrographs were taken with an Olympus MagnaFire digital camera (Olympus, America, Melville, N.Y.).

The specific orange-red fluorescence of ADR was observed in several areas outside the blood brain barrier, including the choroid plexus as previously reported by Bigotte et al. (Bigotte et al., Acta. Neuropathol. 57:121-9 (1982); Bigotte et al., Acta. Neuropathol. 58:193-202 (1982)). ADR fluorescence was clearly distinguishable from the background of untreated control, but not observed in cortex and hippocampus (FIG. 1). When anti-TNF α antibody and ADR were injected into mice together, ADR fluorescence was still observed (FIG. 1B). Fluorescence was not observed in mice treated with anti-TNF α antibody alone (FIG. 1C).

### Example 2

### TNF α Levels in Serum

Example 2 demonstrates that TNF α levels in serum were significantly higher in mice treated with ADR than those from controls.

Mice were treated with 20 mg/kg ADR or saline as control. Blood samples were collected at 1, 3, 6, 9, 24 hours and allowed to clot at 2-8° C. overnight. Serum samples were used to measure TNF α levels, according to the mouse enzyme-linked immunosorbent assay following the manufacturers' instructions (mouse TNF-α/TNFSF1A immunoassay, R&D Systems, Minneapolis, MN). The TNF α concentration in the sample was calculated from a recombinant mouse TNF α standard curve. The minimum detection limit is typically less than 5.1 pg/mL.

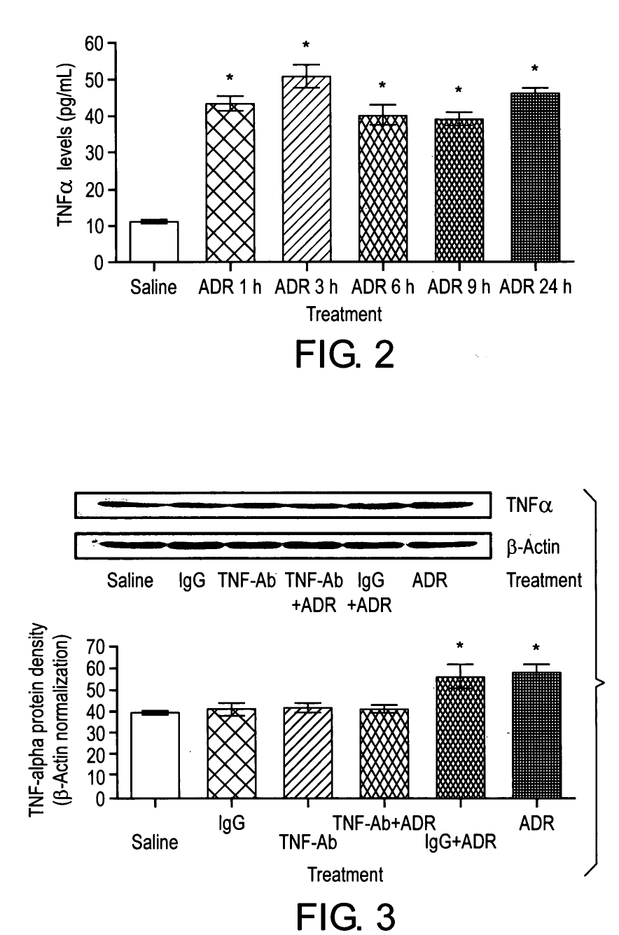

TNF α levels in serum were significantly higher in mice treated with ADR than those from controls (p<0.001, FIG. 2). The increased TNF α levels were detectable as early as 1 hour and were sustained throughout a 24 hour period after ADR treatment.

### Example 3

### TNF α Levels in Brain Tissue

Example 3 demonstrates that TNF α level in brain tissue were significantly increased after administration of ADR.

Brain tissue slices were fixed in 4% paraformaldehyde for 15 minutes, air dried, and washed with PBS. Non-specific proteins were blocked in blocking serum, consisting of 3% normal donkey's serum, and 0.3% Triton x-100 in PBS, and incubated at room temperature for 30 minutes. After blocking, slices were incubated with primary anti-TNF α (Upstate, Lake Placid, N.Y.), and anti-MAP2 antibody (Chemicon, Temecula, Calif.) was used as a neuronal marker, in order to study the location of TNF α in neurons of cortical and hippocampal regions. The sections were kept in a humidified box at 4° C. overnight. Tissues were washed three times with PBS and then were incubated for 1 hour with donkey antibody-conjugated secondary antibodies coated with fluorescent dyes. Excess secondary antibodies were removed by washing three times in PBS and once with deionized H₂O. Tissue slides were mounted with mounting medium (Vectashield, HOURS-100, Vector Laboratories, Burlingame, Calif.). Photomicrographs were obtained using a Leica confocal fluorescence microscope (Leica Microsystems Inc., Bannockburn, Ill., USA).

Brains perfused with PBS were isolated and dissected from four groups of mice 3 hours post injection of a single dose of ADR, anti-TNF α antibody, anti-TNF α antibody immediately followed by ADR, or saline treated mice. Brain was isolated and placed in 0.1 M PBS, pH 7.4 containing protease inhibitors, 4 μg leupeptin, 4 μg pepstatin, and 5 μg aprotinin, washed and minced in ice-cold PBS containing protease inhibitors. Tissues were homogenized with a Dounce homogenizer and centrifuged at 12,500×g at 4° C. for 30 minutes before transferring the supernatant. Protein concentration of brain homogenate was determined by the Bradford assay (Bradford, 1976).

Perfused brain homogenates and isolated mitochondrial proteins were size-separated via 12.5% denaturing polyacrylamide gel electrophoresis (SDS-PAGE) and transferred to a nitrocellulose membrane. The membrane was blocked for 1 hour at room temperature in blocking solution consisting of 5% non-fat dried milk, 0.5% Tween-20 and Tris-buffered saline (TBST), pH 7.9. After blocking, the membrane was incubated overnight at 4° C. with primary antibodies against TNF α (Upstate, Lake Placid, N.Y.) and β-actin (Sigma, St. Louis, Mich.) in homogenate samples, or isolated mitochondria samples for p53 (Ab-11, Oncogene Research, Cambridge, Mass.), Bcl-xS/L (S-18), Bax (P-19) (Santa Cruz Biotechnology, Santa Cruz, Calif.), MnSOD (Upstate, Lake Placid, N.Y.), and glyceraldehyde-3-phosphate dehydrogenase (G-3-PDH, Trevigen, Gaithersburg, Md.) in blocking solution. The membrane was washed twice in TBST and incubated for 1 hour with horseradish peroxidase-conjugated secondary antibodies in blocking solution. After incubation with secondary antibodies, the membrane was washed twice with TBST and once in TBS (TBS without 0.5% Tween-20). Immunoreactivities of the protein bands were detected by enhanced chemiluminescence autoradiography (ECL, Amersham Phamacia Biotech, Arlington Heights, Ill.) as described by the manufacturer.

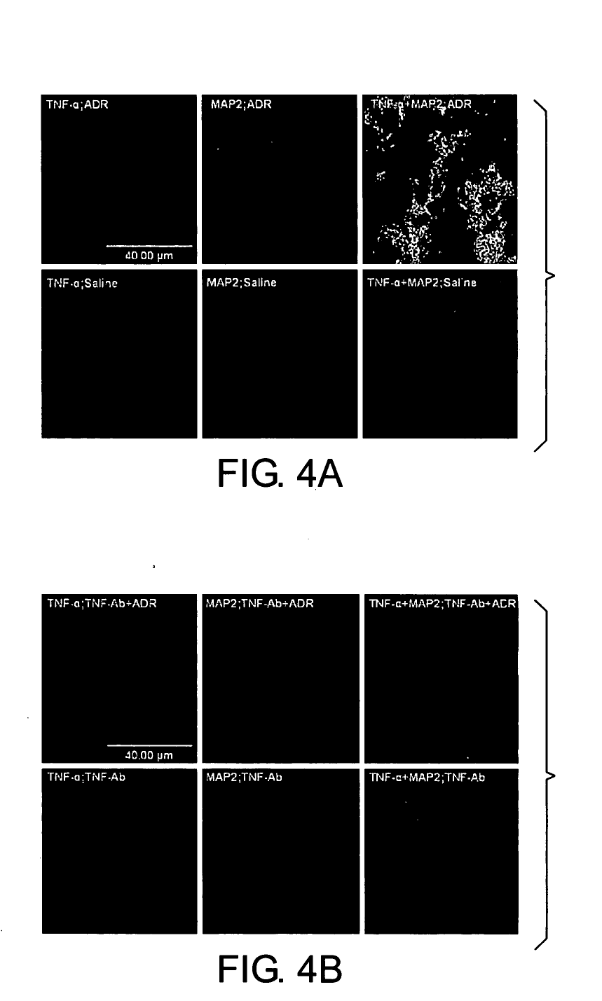

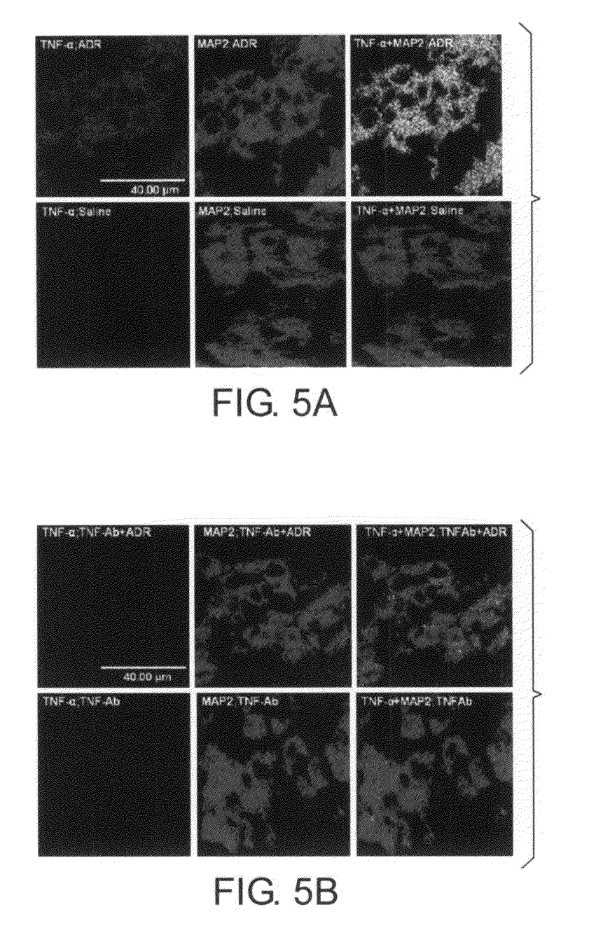

TNF α levels were significantly increased in brain tissue homogenates as detected by Western Blot analysis, following administration of ADR (FIG. 3). To determine the presence of TNF α in brain tissues, tissue slices were stained with anti-TNF α and anti-MAP2 antibodies to localize TNF α. The TNF α levels clearly were increased in neurons of cortex and hippocampus compared with saline controls, since MAP2 is a neuron-specific marker (FIGS. 4 and 5). To verify that the observed TNF α in the brain was mediated by ADR-induced circulating TNF α, a neutralizing antibody against TNF α was co-injected along with ADR. The increased levels of cortical and hippocampal TNF α were blocked in mice treated with anti-TNF α antibody and ADR.

### Example 4

### Effect of ADR on Brain Mitochondrial Function

Example 4 demonstrates that anti-TNF α antibody prevented a decline in mitochondrial respiration of brain tissues.

Mice were perfused via cardiac puncture with cold mitochondrial isolation buffer, the brain promptly removed, the cerebellum dissected away, and mitochondria immediately isolated from the freshly brain by a modification of the method described by Mattiazzi et al. (2002). Brain mitochondria were isolated in cold mitochondrial isolation buffer, containing 0.07 M sucrose, 0.22 M mannitol, 20 mM HEPES, 1 mM EGTA, and 1% bovine serum albumin, pH 7.2. Tissues were homogenized with a Dounce homogenizer and centrifuged at 1,500×g at 4° C. for 5 minutes before transferring the supernatant. The pellets were resuspended and centrifuged at 1,500×g at 4° C. for 5 minutes. The supernatants were combined and centrifuged at 13,500×g at 4° C. for 10 minutes. Mitochondrial pellets were washed twice and finally resuspended in 50-100 μL cold isolation buffer. Protein concentration of isolated mitochondria was determined by the Bradford assay (Bradford, 1976). The final protein concentration was 20-40 mg/ml.

Mitochondrial respiration was determined using Clark-type polarographic oxygen sensors (Hansatech Instruments, Kings Lynn, Norfolk, UK) to measure the rate of oxygen consumption. Freshly isolated mitochondria were suspended in respiration buffer at a concentration of 0.5 mg mitochondrial protein per mL of respiration buffer, which consists of 0.25 M sucrose, 50 mM HEPES, 1 mM EGTA, 10 mM KH₂PO₄, and 2 mM MgCl₂, pH 7.4. Oxygen consumption was measured with either pyruvate (10 mM) plus malate (10 mM) or succinate (10 mM) as substrates for respiration from complex I or complex II in the absence of exogenous ADP (state II) and after addition of 300 mM ADP (state III respiration). Rotenone (5 μM) was added to the reaction to inhibit respiration from complex I when succinate was used as the substrate. The ATPase inhibitor oligomycin (100 μg/mL) was added to inhibit mitochondrial respiration such that state IV respiration was similar to the state II respiration rate. FCCP (1 μM), an uncoupling agent, was added as a control of respiration. Respiration control ratios (RCR) were calculated as the ratios of state III and state II respiration as described previously by Estabrook (1967). The unit for state II rate and state III rate is nmole/min/mg protein.

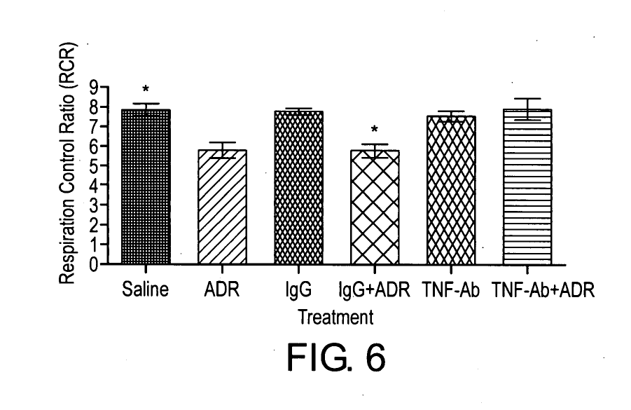

To investigate the effect of ADR on brain mitochondrial function, brain mitochondrial respiration using pyruvate plus malate and succinate as the substrates was measured. The data are presented as the RCR of each treatment group and the saline-treated control group from each set of experiments. As shown in FIG. 6, the values of RCR from state III and state II respiration were significantly decreased in the ADR treatment group compared to controls (p<0.05).

In mice treated with anti-TNF α antibody only, or in mice treated with anti-TNF α antibody followed by ADR, brain mitochondrial respiration was not significantly different from that of the control group. Taken together, these results indicate that ADR-induced circulating TNF α levels subsequently increased brain levels of TNF α, which led to inhibition of the NAD-linked state III respiration rate. The latter is mediated through complex I. The anti-TNF α antibody prevented the decline in mitochondrial respiration of brain tissues, consistent with this notion.

### Example 5

### Effect of Anti-TNF α Antibody on p53 and Bcl-xL Complex in Mitochondria

Example 5 demonstrates that blocking circulating TNF α with anti-TNF α antibody resulted in no ADR-mediated increased p53 and Bcl-xL complex in mitochondria.

Isolated mitochondrial protein (500 μg) was resuspended in 500 μL RIPA buffer (9.1 mM Na₂HPO₄, 1.7 mM NaH₂PO₄, 150 mM NaCl, 0.5% sodium deoxycholate, 1% v/v Nonidet P40, 0.1% sodium dodecyl sulfate, pH 7.2). Protease inhibitors (0.1 mg PMSF and 1 μg aprotinin per mL RIPA) were added at the time of use followed by incubation with 5 μg/mL of mouse p53 antibody (Ab-11, Oncogene) at 4° C. overnight. Protein A/G-Agarose (50 μL) was added to the reaction mixture with the antibody. Immunocomplexes were collected by centrifugation at 1,000×g at 4° C. for 5 minutes, then washed four times with RIPA buffer. Immunoprecipitated samples were recovered by resuspending in 2×sample loading buffer, and p53 or Bcl-xL (Santa Cruz Biotechnology, Santa Cruz, Calif.) proteins were detected by Western blot. Immunoreactivity was evaluated on immunoblots by densitrometric analysis using a Bio-RAD densitometer (Bio-RAD Laboratory, Inc, Hercules, Calif., USA).

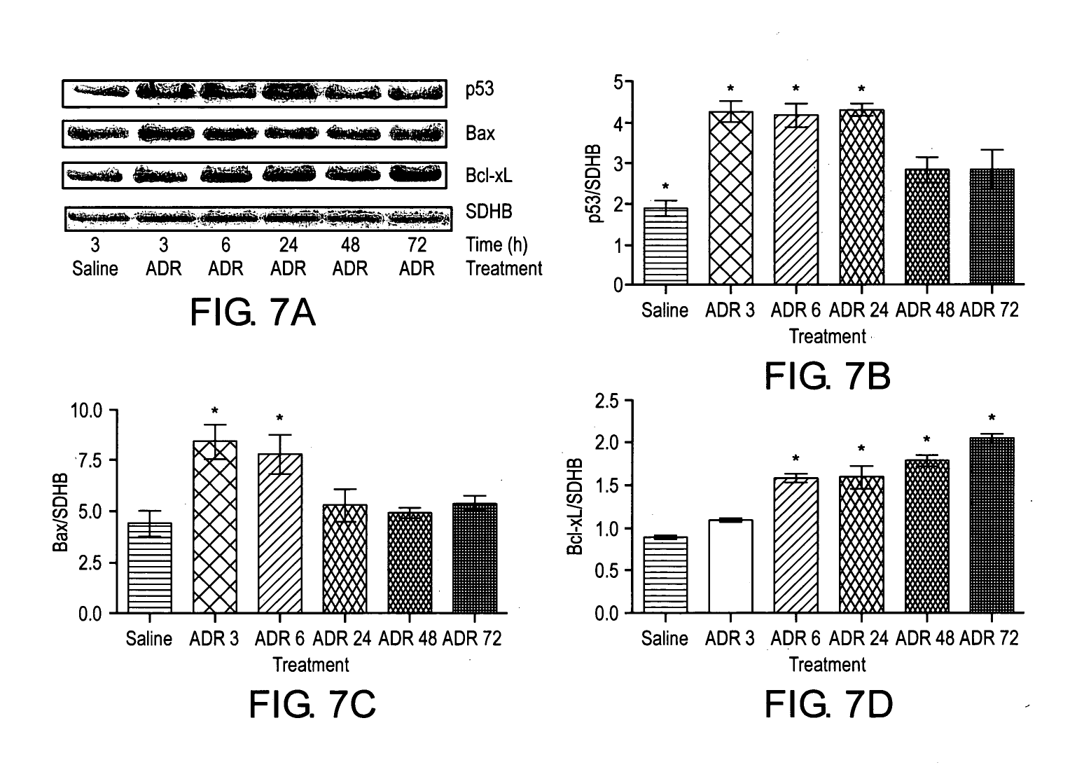

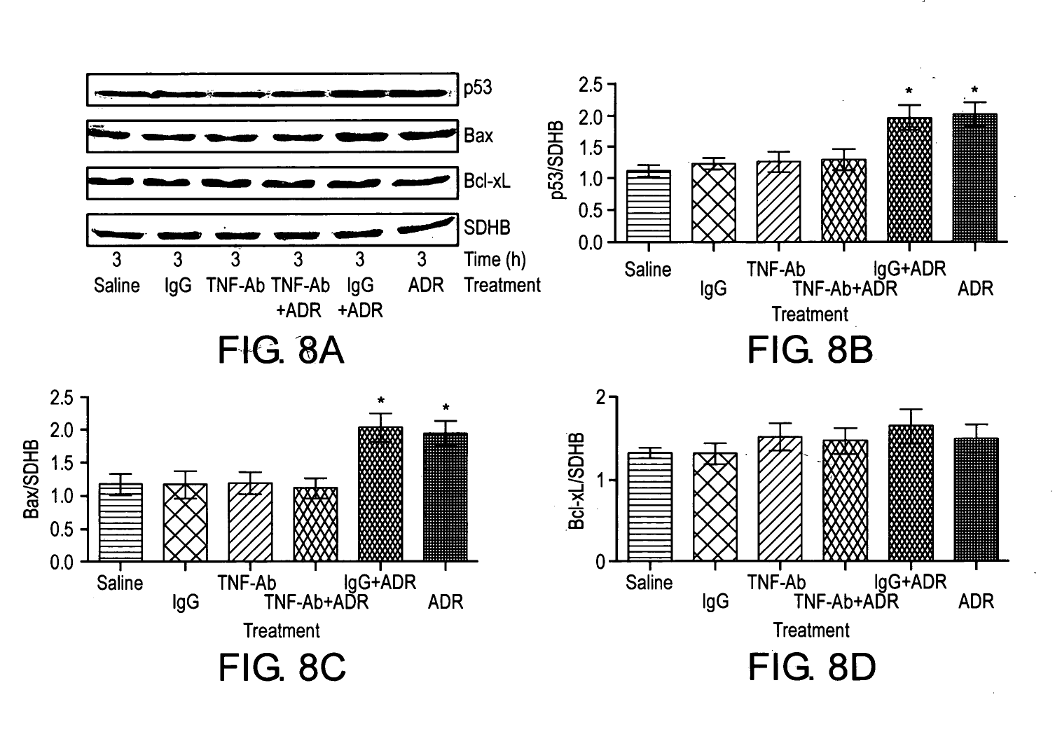

To probe the possibility that ADR induced increased levels of serum and brain TNF α are associated with altered levels of pro- and anti-apoptotic proteins in brain mitochondria, the levels of the pro-apoptotic proteins, p53, Bcl-xS, and Bax, and the anti-apoptotic proteins, Bcl-xL, and MnSOD, were quantified (FIG. 7). The results demonstrate that p53 was increased in mitochondria early times after ADR treatment (3, 6, 24 hours), then declined at 48, and 72 hours. Bax increased with a similar kinetics to that of p53. The anti-apoptotic protein, Bcl-xL increased after treatment with ADR at 6, 24, 48 and 72 hours. There were no significant changes in the levels of MnSOD and Bcl-xS at any time point studied. The level of G-3-PDH was not changed and was used as loading control for normalization. These results demonstrated a rapid increase of pro-apoptotic proteins and anti-apoptotic Bcl-xL in the mitochondria of ADR treated mice, which are associated with increased TNF α in the brain. Consistent with the results shown above, blocking ADR-mediated elevated TNF α with anti-TNF α antibody resulted in no increase of pro-apoptotic proteins in mitochondria 3 hours after treatment with ADR (FIG. 8).

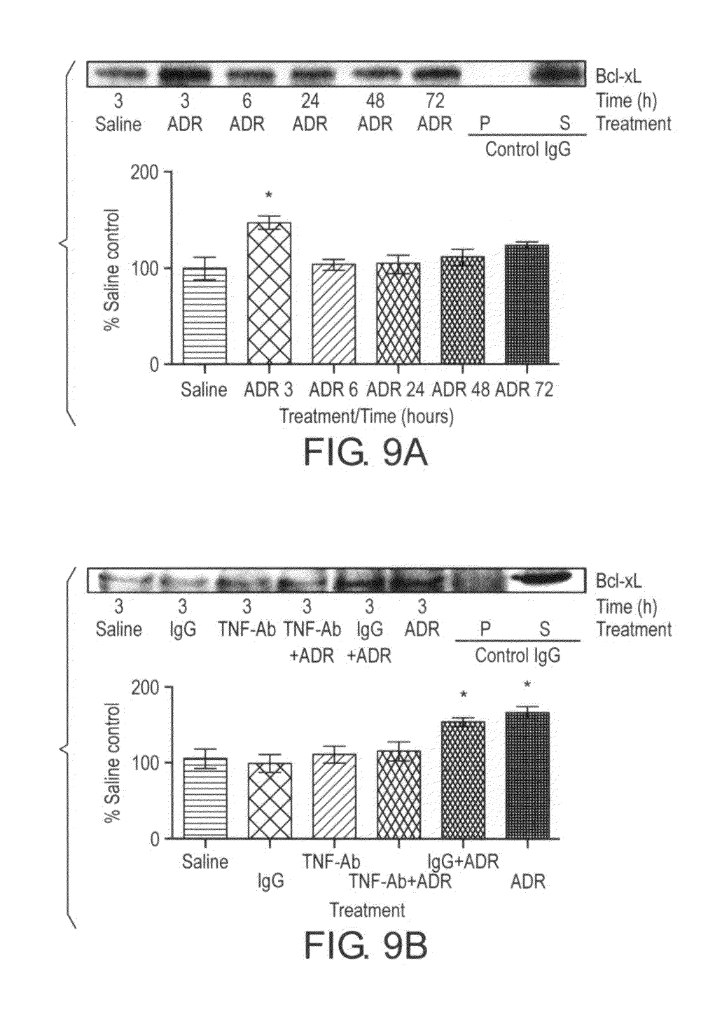

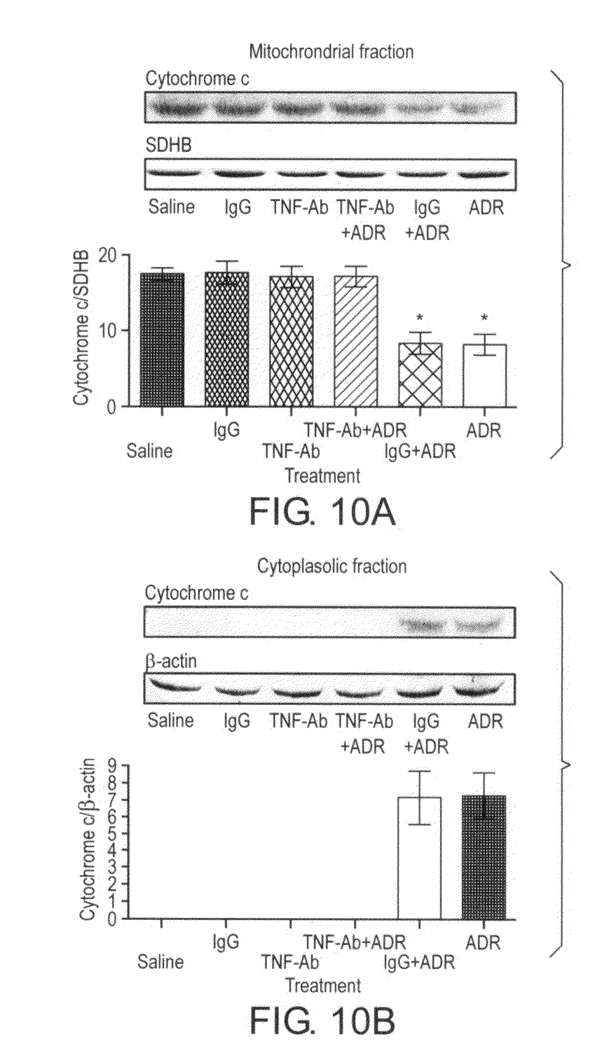

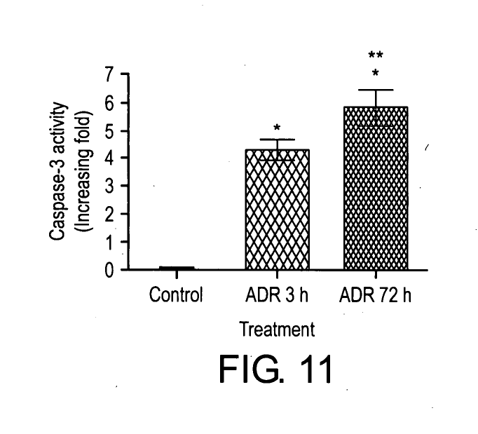

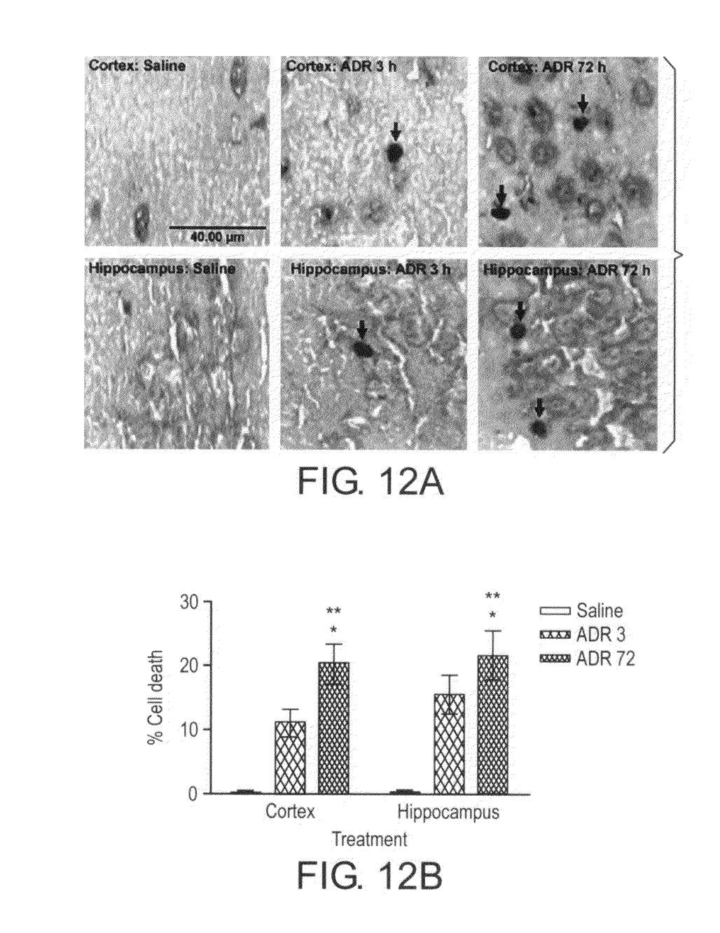

p53 can participate in induction of apoptosis by acting directly at the mitochondria (Marchenko et al., J. Biol. Chem. 275:16202-12 (2000); Mihara et al., Molecular Cell 11:577-90 (2003)). Localization of p53 to mitochondria occurs in response to apoptotic signals and precedes cytochrome C release and procaspase-3 activation (Schuler et al., Biochem. Soc. Trans. 29:684-8 (2001). To determine the ability of p53 to interact with Bcl-xL in brain mitochondria, immunoprecipitation was performed using an antibody to p53 to precipitate mitochondrial proteins, and the complexes were probed with antibodies to p53 and Bcl-xL by Western blot analysis. The results showed specific increases of p53 and Bcl-xL in the ADR treatment groups compared to the controls (FIG. 9A). Blocking circulating TNF α with anti-TNF α antibody resulted in no ADR-mediated increased p53 and Bcl-xL complex in mitochondria (FIG. 9B).

### Example 6

### Adriamycin-Mediated Nitration of Manganese Superoxide Dismutase

Example 6 demonstrates that treatment with ADR leads to an increase in circulating level of TNF-α in wild-type mice and in mice deficient in the inducible form of nitric oxide (iNOSKO) but only causes a decline in mitochondrial respiration and mitochondrial protein nitration after only in wild-type mice and not the iNOSKO mice.

8-week-old male B6C3 mice (25-30 g) were kept under standard conditions. Mice were injected with a single intraperitoneal (i.p.) treatment with adriamycin (doxorubicin hydrochloride, 20 mg/kg), TNF-α, anti-mouse TNF-antibody (40 ng/kg), dipropylenetriamine NONOate (DPTA NONOate, 4 mm), lipopolysaccharide (LPS 1 mg/kg), NG-nitro-L-arginine-methyl esther (L-NAME, 10 mg/kg) or pre-immune IgG.

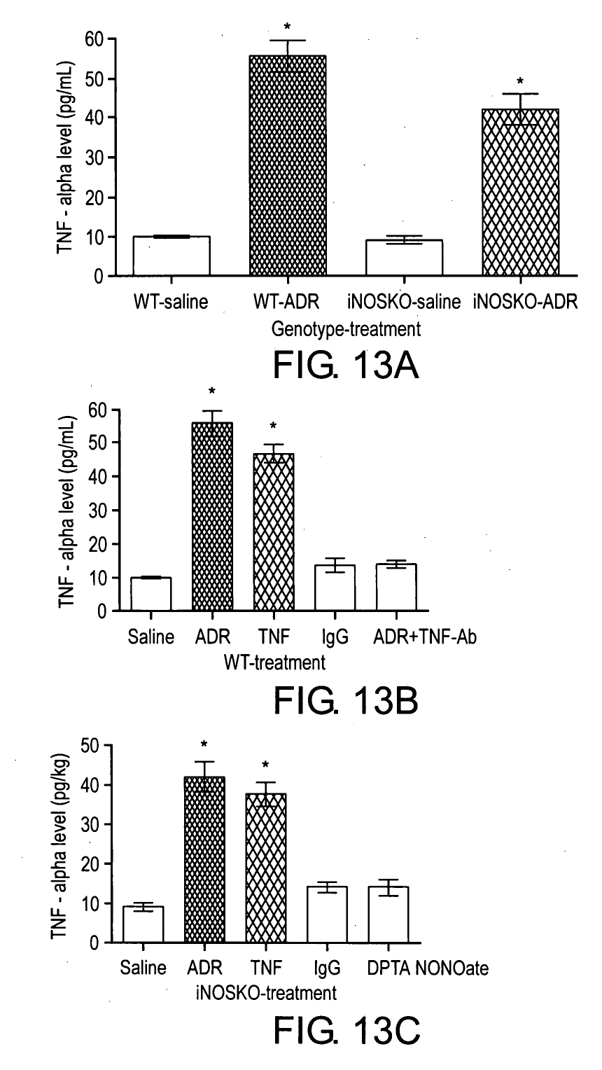

Mice were treated with 20 mg/kg ADR, TNF-α, anti-mouse TNF-antibody, dipropylenetriamine NONOate, lipopolysaccharide, NG-nitro-L-arginine-methyl esther or pre-immune IgG, as well as saline as control. Blood samples were collected at 3 hours after ADR treatment and allowed to clot at 2-8° C. overnight. Serum samples were used to measure TNF levels, according to the mouse enzyme-linked immunosorbent assay following the manufacturers' instructions (mouse TNF-α/TNFSF1A immunoassay, R&D Systems, Minneapolis, Minn.). The TNF concentration in the sample was calculated from a recombinant mouse TNF standard curve. The minimum detection limit is typically less than 5.1 pg/mL. The results demonstrate that DPTA NONOate did not lead to changes in iNOSKO mice serum TNF-α levels (FIG. 13).

Mice were perfused via cardiac puncture with cold mitochondrial isolation buffer, the brain promptly removed, the cerebellum dissected away, and mitochondria immediately isolated from the freshly brain by a modification of the method described by Mattiazzi et al. (2002). Brain mitochondria were isolated in cold mitochondrial isolation buffer, containing 0.07 M sucrose, 0.22 M mannitol, 20 mM HEPES, 1 mM EGTA, and 1% bovine serum albumin, pH 7.2. Tissues were homogenized with a Dounce homogenizer and centrifuged at 1,500×g at 4° C. for 3 minutes before transferring the supernatant fluid. The pellets were resuspended and centrifuged at 1,500×g at 4° C. for 3 minutes. The supernatant fluids were combined and centrifuged at 1,500×g at 4° C. for 3 minutes. These supernatant fluids were then centrifuged at 13,500×g at 4° C. for 10 minutes. Mitochondrial pellets were washed twice and finally resuspended in 50-100 μL cold isolation buffer. Protein concentration of isolated mitochondria was determined by the Bradford assay (Bradford, 1976).

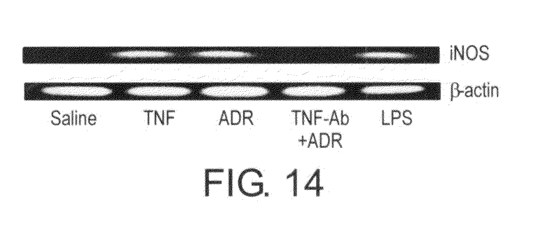

The possibility that ADR caused elevation of circulating TNF-induced iNOS expression in brain tissues was investigated. As shown in FIG. 14, the levels of iNOS mRNA were significantly increased in TNF-, ADR-, and LPS-treated mice compared to saline controls. Neutralizing antibody against circulating TNF prevented TNF-induced iNOS mRNA expression in brain tissues.

Mitochondrial respiration was determined using Clark-type polarographic oxygen sensors (Hansatech Instruments, UK) to measure the rate of oxygen consumption. Freshly-isolated mitochondria were suspended in respiration buffer at a concentration of 0.5 mg mitochondrial protein/mL respiration buffer, which consisted of 0.25 M sucrose, 50 mM HEPES, 1 mM EGTA, 10 mM KH₂PO₄, 2 mM MgCl₂ and 0.2% BSA, pH 7.4. Oxygen consumption was measured with either pyruvate (10 mM) or pyruvate plus malate (10 mM) as substrates for respiration from complex I, in the absence of exogenous ADP (state II), and after addition of 300 mM ADP (state III respiration). The ATPase inhibitor, oligomycin (100 μg/mL), was added to inhibit mitochondrial respiration such that state IV respiration was similar to the state II respiration rate. FCCP (1 μM), an uncoupling agent, was added as a control respiration. Respiration control ratios (RCR) were calculated as the ratios of state III and state II respiration. The unit for state II rate and state III rate is nanomoles per minute per milligram of protein. These results demonstrated that ADR-induced circulating TNF levels subsequently increased brain levels of TNF, which may activate iNOS to produce NO resulting in the inhibition of the NAD-linked state III respiration rate.

Brains were perfused, the cerebellum removed and the brain dissected from mice treated with i.p. ADR 20 mg/kg, or saline as a control, for 3 hours. Brains were placed in ice-cold 50 mM PBS, pH 7.4, containing the protease inhibitors 4 μg leupeptin, 4 μg pepstatin, and 5 μg aprotinin, washed and chopped in ice-cold PBS containing protease inhibitors. Tissues were homogenized with a Potter-Elvehjem glass homogenizer with a loose fitting Teflon® pestle, and protein concentration was determined by the Bradford assay (Bradford, 1976).

MnSOD activities in the brain homogenates were measured by the nitroblue tetrazolium (NBT)-bathocuproin sulfonate (BCS) reduction inhibition method, as described (Spitz and Oberley 1989). Sodium cyanide (2 mM) was used to inhibit copper-zinc superoxide dismutase (Cu/ZnSOD) activity. MnSOD activity was expressed in units per milligram of protein.

Brain homogenate proteins were size-separated using denaturing sodium dodecyl sulfate-polyacrylamide gel electrophoresis (SDS-PAGE). Proteins (50 μg) were electrophoresed on 12.5% SDS-PAGE and transferred to a nitrocellulose membrane. The membrane was blocked for 1 hour at room temperature in blocking solution consisting of 5% non-fat dried milk, Tris-buffered saline (TBST; 10 mM Tris-HC, 150 mM NaCl, 0.05% Tween 20), pH 7.9. After blocking, the membrane was incubated overnight at 4° C. with primary antibodies against MnSOD (Upstate, Lake Placid, N.Y., USA), Cu/ZnSOD (Calbiochem, San Diego, Calif., USA) and β-actin (Sigma) in blocking solution. The membrane was washed twice in TBST, then incubated for 1 hour with horseradish peroxidase-conjugated secondary antibody in blocking solution. After incubation with secondary antibody, the membrane was washed twice with TBST and once in TBS (TBS without 0.05% Tween-20). Immunoreactivities of the protein bands were detected by enhanced chemiluminescence autoradiography (ECL; Amersham Phamacia Biotech, Arlington Heights, Ill., USA) as described by the manufacturer.

Solubilized isolated mitochondrial proteins (500 μg) in 500 μL RIPA buffer (9.1 mM Na₂HPO₄, 1.7 mM NaH₂PO₄, 150 mM NaCl, 0.5% sodium deoxycholate, 1% v/v Nonidet P40, 0.1% SDS, pH 7.2) were incubated for 16 hours at 4° C. with 10 μg/mL anti-nitrotyrosine antibody (Cayman Chemical, Ann Arbor, Mich., USA) or polyclonal anti-MnSOD antibody (Upstate Biotechnology). Immune complexes were precipitated with 50 μL protein A/G-Agarose. Immunocomplexes were collected by centrifugation at 1500 g for 5 minutes at 4° C., and then washed four times with RIPA buffer. Immunoprecipitated samples were recovered by resuspending in 50 μL 2×sample loading buffer, heated to 95° C. for 5 minutes, and immediately fractionated by reducing SDS-PAGE in 12.5% gels. Isolated mitochondria samples from mouse brains were treated with 30 μM peroxynitrite as positive control (provided by Dr. Timothy R. Miller, Graduate Center for Toxicology, University of Kentucky, Lexington, Ky., USA) and the sample was incubated with 10 μg/mL IgG as negative control. Immunoreactivities of the MnSOD nitrated protein bands were detected by ECL (Amersham Phamacia Biotech) as described by the manufacturer. Nitration of MnSOD was quantified using an akaline phosphatase-linked secondary antibody (Sigma), as described previously (Sultana et al. 2004), and evaluated by densitometric analysis used a Bio-Rad densitometer (Bio-Rad Laboratories, Hercules, Calif., USA).

Supernatant fluids obtained from the control- and ADR-treated mitochondria, after immunoprecipitation of MnSOD, were dissolved in rehydration buffer containing 8 M urea, 2 M thiourea, 2% 3-[(3-cholamidopropyl)dimethylammonio]-1-propanesulfonate (CHAPS), 0.2% (v/v) biolytes, 50 mM dithiothreitol (DTT) and bromophenol blue. The samples were sonicated three times for 20 s on ice, and then applied to the IPG Readystrips (pH 3-10) from Bio-Rad to obtain gel maps (Levine et al. 1994). Isoelectric focusing was carried out at 20° C. as follows: 800 V for 2 hours linear gradient; 1200 V for 4 hours of slow gradient; 8000 V for 8 hours of linear gradient; 8000 V for 10 hours of rapid gradient. The strips were stored at −80° C. until second dimension separation was performed. Gel strips were equilibrated, before second dimension separation, for 10 minutes in 50 mM Tris-HCl (pH 6.8) containing 6 M urea, 1% (m/v) SDS, 30% (v/v) glycerol and 0.5% DTT, followed by re-equilibrium for 10 minutes in the same buffer containing 4.5% iodoacetamide in place of DTT. The gel strips were then placed in the linear gradient precast Criterion Tris-HCl gels (8-16%, Bio-Rad) to perform the second dimension electrophoresis.

The gels were fixed in a solution containing 10% (v/v) methanol and 7% (v/v) acetic acid for 50 minutes, and then stained overnight at room temperature with gentle agitation in 50 mL Sypro ruby gel stain (Bio-Rad). The gels were placed in deionized water overnight and scanned.

3-Nitrotyrosine levels, a peroxynitrite reaction product marker, were determined as described previously (Lauderback et al. 2001). Briefly, 5 μg isolated mitochondrial proteins were incubated with Laemmli sample buffer (0.125 M Trisma base, pH 6.8, 4% SDS, 20% glycerol) for 20 minutes, and then 250 ng protein were blotted onto the nitrocellulose membrane using a slot-blot apparatus. The membrane was rinsed with TBST buffer, blocked by incubation in the presence of 5% BSA, followed by incubation with rabbit polyclonal anti-nitrotyrosine antibody as primary antibody for 1 h. The membranes were washed with TBST buffer and further incubated with goat anti-rabbit horseradish peroxidase-conjugated secondary antibody for 1 h. After incubation with secondary antibody, the membrane was washed twice with TBST and once in TBS (TBS without 0.05% Tween-20). Immunoreactivities of the protein bands were detected by ECL (Amersham Phamacia Biotech) as described by the manufacturer. Blots were scanned with Adobe Photoshop and quantified by the densitometry method (Bio-Rad).

At 3 hours after treatment, mice were anesthetized and perfused via cardiac puncture with 0.1 M PBS, pH 7.4. The MRNA was isolated using the Micro-FastTrack 2.0 Kit (Invitrogen, Groningen, Netherlands) according to the manufacturer's instructions. The purified MRNA (5 μg) was subjected to reverse-transcription into first strand cDNA in each 20 μL of reaction mixture using the SuperScript III First-Strand Synthesis System for RT-PCR (Invitrogen) according to the manufacturer's instruction. A 25 μL PCR reaction contained 5 μL first strand cDNA, 0.1 U Taq DNA polymerase (Invitrogen), 10 mM 10×PCR buffer, 1.5 mM MgCl₂, 2.5 mM dNTP and 10 pmol of each specific primer. PCR samples were subjected to the following conditions: 35 cycles for iNOS (94° C.: 1 minute; 65° C.: 1 minute; 72° C.: 1 minute), 32 cycles for β-actin (94° C.: 30 minutes; 55° C.: 30 s; 72° C.: 10 minutes), on a thermocycle (Perkin-Elmer). The primers with the target gene sequences were synthesized by Invitrogen. The primer sequence iNOS was 5′-CTGATGGTCAAGATCCAG-A-GGTCT-3′ (forward) and 5′-CTGCATGTGCTTCATGAAGGA-CTCT-3′(reverse), and the primer for β-actin was 5′-TGTTAC-CAACTGGGACGACA-3′ (forward) and 5′-CTGGGTCATCTT-TTCACCGT-3′ (reverse). After amplification, PCR products were subjected to 1% agarose gel electrophoresis, and visualized by ethidium bromide staining. The relative density of bands was analyzed under ultraviolet light (Bio-Rad). Experiments were repeated three times for reproducibility.

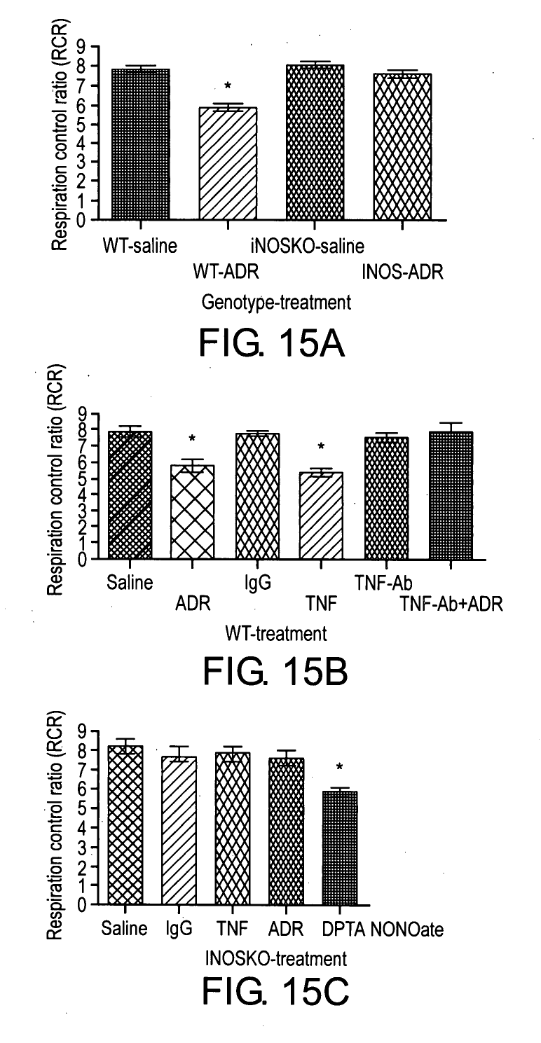

To investigate the effect of ADR on brain mitochondrial function, mitochondrial respiration using pyruvate plus malate as the substrates was measured. The data are presented as the RCR of each treatment group and the saline-treated control group from each set of experiments. The values of RCR in mitochondria from wild-type mice, but not iNOSKO mice, were significantly decreased in the ADR-treated group compared with the control saline group (p<0.05, (FIG. 15a)). Treatment of wild-type mice with TNF caused a mitochondrial respiration decline similar to that observed by ADR treatment (FIG. 15b). Injection of neutralizing antibody to TNF followed by ADR abolished the reduced brain mitochondrial respiration (FIG. 15b). Importantly, in iNOSKO mice, treatment with TNF or ADR had no effect on the RCR, while treatment with DPTA NONOate, a NO generator, significantly lowered the RCR in iNOSKO mice (p<0.05, (FIG. 15c)). These results suggest that ADR-induced circulating TNF levels subsequently increased brain levels of TNF, which may activate iNOS to produce NO resulting in the inhibition of the NAD-linked state III respiration rate.

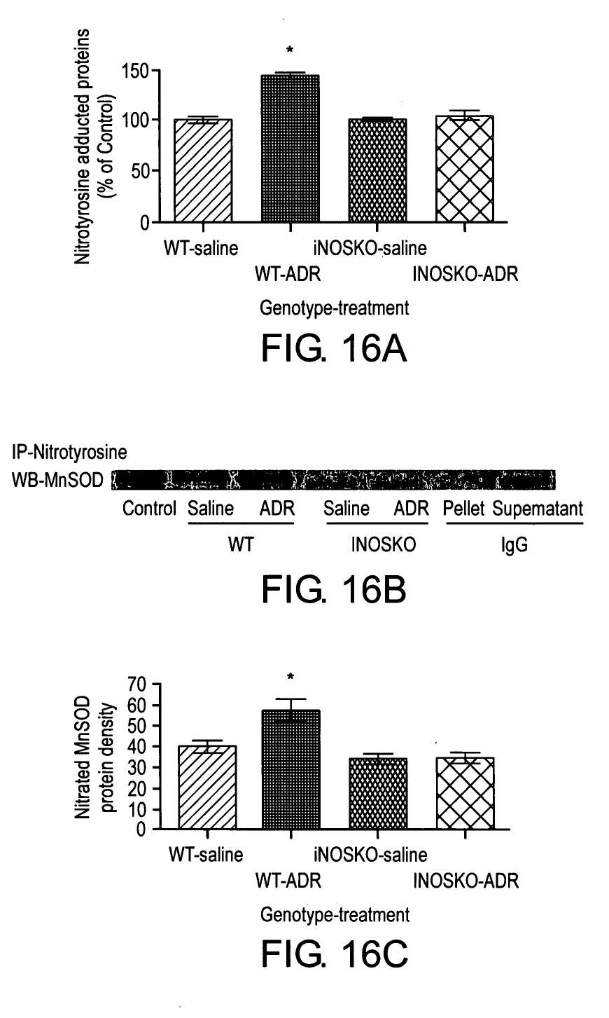

As reactive products of NO can cause brain protein nitration (Butterfield and Stadtman 1997; Castegna et al. 2003; Sultana et al. 2006), nitrotyrosine adducted proteins from mitochondria, isolated form brain of mice 3 hours after treatment with ADR at 20 mg/kg were determined. The levels of tyrosine-nitrated proteins were significantly increased in wild-type mice after treatment with ADR for 3 hours (p<0.01, (FIG. 16a)), but not in iNOSKO mice.

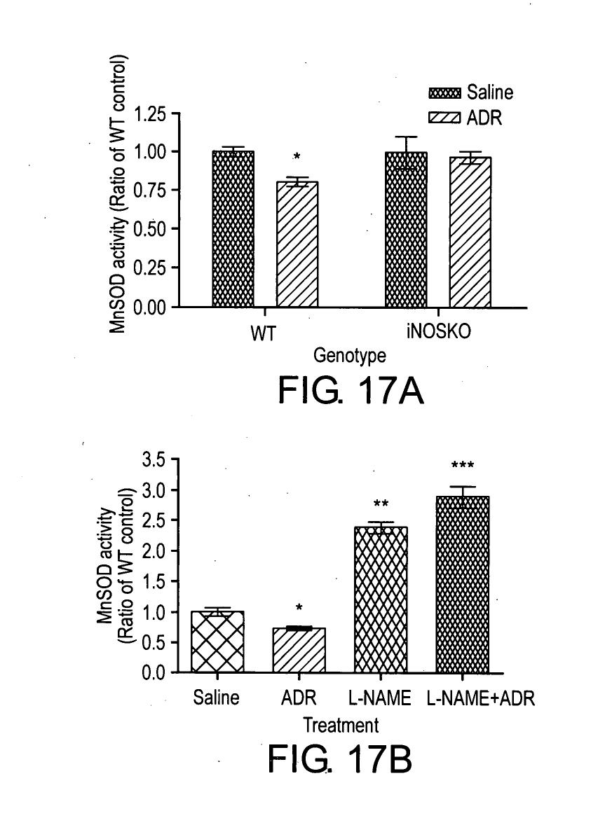

An increased NO level within the mitochondria could lead to nitration of MnSOD. Immunoprecipitation of mitochondrial protein with anti-nitrotyrosine antibody with anti-MnSOD antibody was used to pull down nitrotyrosine-MnSOD immunocomplex. The immunoprecipitated protein demonstrated an increase of nitrated MnSOD in wild-type mice (p<0.01, (FIGS. 16b and c)), but not in iNOSKO mice. As MnSOD is known to be sensitive to peroxynitrite-induced inactivation, MnSOD activity after treatment with ADR at 20 mg/kg for 3 hours was determined. MnSOD activity was significantly decreased by ADR in brain tissue homogenates of wild-type mice, but no in those of iNOSKO mice (p<0.01, (FIG. 17a)). Treatment of mice with L-NAME, a non-selective NOS inhibitor, prevented MnSOD inactivation and led to increased MnSOD activity in brains compared with those of saline- or ADR-treated mice (p<0.001, (FIG. 17b)).

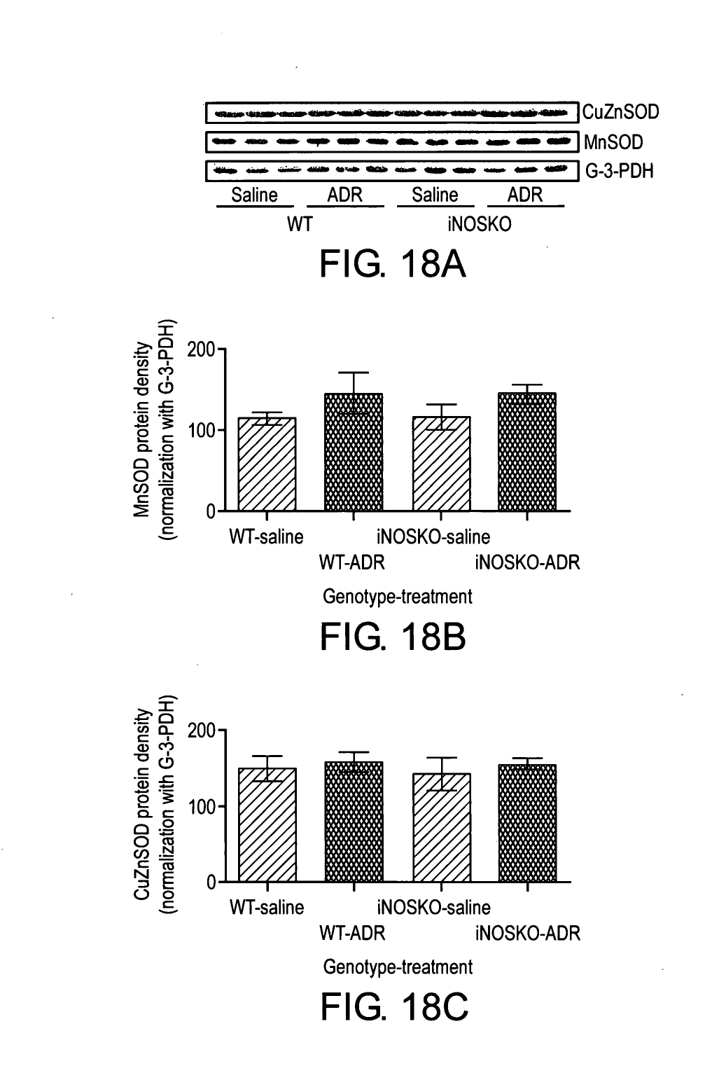

There is a possibility that the decline in MnSOD activity in brains from ADR-treated wild-type mice is due to a reduced MnSOD protein level. Accordingly, it was determined the MnSOD and CuZnSOD levels in brain tissue homogenates by western blot analysis following administration of ADR. The results demonstrated no change in levels of either MinSOD or CuZnSOD in both wild-type and iNOSKO mouse brain (FIGS. 18a and b).

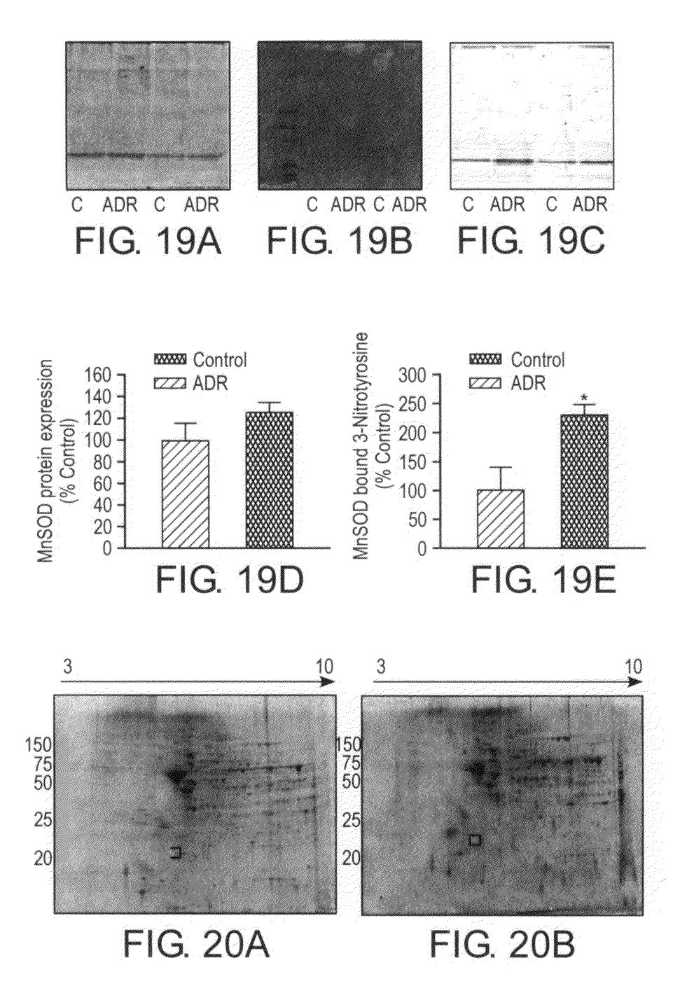

Further, to confirm the correct identification of MnSOD as a nitrated protein, immunoprecipitated MnSOD protein from control and ADR-treated mitochondria was probed with anti-3-nitrotyrosine antibody. This showed a significant increase in nitration of MnSOD (p<0.04) and no difference in expression (FIGS. 19a and b). In addition, the 2D map obtained from the supernatant fluid of the MnSOD immunoprecipitated sample (FIG. 20b) showed a missing spot corresponding to MnSOD (FIGS. 19a and b). These results demonstrate the nitration of MnSOD in ADR-treated mitochondria.

Patents, patent applications, publications, scientific articles, books, web sites, and other documents and materials referenced or mentioned herein are indicative of the levels of skill of those skilled in the art to which the inventions pertain. All claims in this application, and all priority applications, including but not limited to original claims, are hereby incorporated in their entirety into, and form a part of, the written description of the invention. Applicants reserve the right to physically incorporate into this specification any and all materials and information from any such patents, applications, publications, scientific articles, web sites, electronically available information, and other referenced materials or documents. Applicants reserve the right to physically incorporate into any part of this document, including any part of the written description, and the claims referred to above including but not limited to any original claims.

The inventions have been described broadly and generically herein. Each of the narrower species and subgeneric groupings falling within the generic disclosure also form part of these inventions. This includes the generic description of each invention which hereby include, including any claims thereto, a proviso or negative limitation removing or optionally allowing the removal of any subject matter from the genus, regardless of whether or not the excised materials or options were specifically recited or identified in haec verba herein, and all such variations form a part of the original written description of the inventions

The inventions illustratively described and claimed herein can suitably be practiced in the absence of any element or elements, limitation or limitations, not specifically disclosed herein or described herein as essential. Thus, for example, the terms “comprising,” “including,” “containing,” “for example,” etc., shall be read expansively and without limitation. As used herein and in the appended claims, the singular forms “a,” “an,” and “the” include plural reference unless the context clearly dictates otherwise.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement was specifically and without qualification or reservation expressly adopted by Applicants in a responsive writing specifically relating to the application that led to this patent prior to its issuance.

The terms and expressions employed herein have been used as terms of description and not of limitation, and there is no intention in the use of such terms and expressions, or any portions thereof, to exclude any equivalents now know or later developed, whether or not such equivalents are set forth or shown or described herein or whether or not such equivalents are viewed as predictable, but it is recognized that various modifications are within the scope of the invention claimed, whether or not those claims issued with or without alteration or amendment for any reason. Thus, it shall be understood that, although the present invention has been specifically disclosed by preferred embodiments and optional features, modifications and variations of the inventions embodied therein or herein disclosed can be resorted to by those skilled in the art, and such modifications and variations are considered to be within the scope of the inventions disclosed and claimed herein.

Specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. Where examples are given, the description shall be construed to include but not to be limited to only those examples. It will be readily apparent to one skilled in the art that varying substitutions and modifications may be made to the invention disclosed herein without departing from the scope and spirit of the invention, and from the description of the inventions, including those illustratively set forth herein, it is manifest that various modifications and equivalents can be used to implement the concepts of the present invention without departing from its scope. A person of ordinary skill in the art will recognize that changes can be made in form and detail without departing from the spirit and the scope of the invention. The described embodiments are to be considered in all respects as illustrative and not restrictive. Thus, for example, additional embodiments are within the scope of the invention and within the following claims.

We claim:

1. A method of treating toxic effects of chemotherapeutic drugs on the central nervous system comprising administering a therapeutically effective amount of anti-Tumor Necrosis Factor-α antibody to a patient in need thereof to alleviate symptoms associated with treatment with chemotherapeutic drugs.

2. The method of claim 1 wherein the chemotherapeutic drug is Adriamycin.

3. The method of claim 1 wherein the anti-Tumor Necrosis Factor-α antibody is etanercept.

4. The method of claim 1 wherein the anti-Tumor Necrosis Factor-α antibody is infliximab.

5. The method of claim 1 wherein the anti-Tumor Necrosis Factor-α antibody is adalimumab.

6. The method of claim 1 wherein the anti-Tumor Necrosis Factor-α antibody is any humanized monoclonal antibody specific for Tumor Necrosis Factor-α.

7. The method of claim 1 wherein the anti-Tumor Necrosis Factor-α antibody is any molecule that contains extracellular portions of a human Tumor Necrosis Factor-α receptor.

8. A method of decreasing serum level of Tumor Necrosis Factor-α comprising administering anti-Tumor Necrosis Factor-α antibody to a patient in need thereof in a therapeutic amount effective to alleviate toxic effects of chemotherapeutic drugs on the central nervous system.

9. The method of claim 8 wherein the chemotherapeutic drug is Adriamycin.

10. The method of claim 8 wherein the anti-Tumor Necrosis Factor-α antibody is etanercept.

11. The method of claim 8 wherein the anti-Tumor Necrosis Factor-α antibody is infliximab.

12. The method of claim 8 wherein the anti-Tumor Necrosis Factor-α antibody is adalimumab.

13. The method of claim 8 wherein the anti-Tumor Necrosis Factor-α antibody is any humanized monoclonal antibody specific for Tumor Necrosis Factor-α.

14. The method of claim 8 wherein the anti-Tumor Necrosis Factor-α antibody is any molecule that contains extracellular portions of a human Tumor Necrosis Factor-α receptor.

15. A method of preventing decline in mitochondrial respiration of brain tissues caused by the use of chemotherapeutic drugs, comprising administering anti-Tumor Necrosis Factor-α antibody to a patient in need thereof in a therapeutic amount effective to alleviate toxic effects of chemotherapeutic drugs on the central nervous system.

16. The method of claim 15 wherein the chemotherapeutic drug is Adriamycin.

17. The method of claim 15 wherein the anti-Tumor Necrosis Factor-α antibody is etanercept.

18. The method of claim 15 wherein the anti-Tumor Necrosis Factor-α antibody is infliximab.

19. The method of claim 15 wherein the anti-Tumor Necrosis Factor-α antibody is adalimumab.

20. The method of claim 15 wherein the anti-Tumor Necrosis Factor-α antibody is any humanized monoclonal antibody specific for Tumor Necrosis Factor-α.

21. The method of claim 15 wherein the anti-Tumor Necrosis Factor-α antibody is any molecule that contains extracellular portions of a human Tumor Necrosis Factor-α receptor.

22. A method of preventing p53 translocation to mitochondria comprising administering anti-Tumor Necrosis Factor-α antibody to a patient in need thereof in a therapeutic amount effective to alleviate toxic effects of chemotherapeutic drugs on the central nervous system.

23. The method of claim 22 wherein the chemotherapeutic drug is Adriamycin.

24. The method of claim 22 wherein the anti-Tumor Necrosis Factor-α antibody is etanercept.

25. The method of claim 22 wherein the anti-Tumor Necrosis Factor-α antibody is infliximab.

26. The method of claim 22 wherein the anti-Tumor Necrosis Factor-α antibody is adalimumab.

27. The method of claim 22 wherein the anti-Tumor Necrosis Factor-α antibody is any humanized monoclonal antibody specific for Tumor Necrosis Factor-α.

28. The method of claim 22 wherein the anti-Tumor Necrosis Factor-α antibody is any molecule that contains extracellular portions of a human Tumor Necrosis Factor-α receptor.

29. A method of alleviating the symptoms of somnolence comprising administering anti-Tumor Necrosis Factor-α antibody to a patient in need thereof in a therapeutic amount effective to a patient to alleviate toxic effects of chemotherapeutic drugs on the central nervous system.

30. The method of claim 29 wherein the chemotherapeutic drug is Adriamycin.

31. The method of claim 29 wherein the anti-Tumor Necrosis Factor-α antibody is etanercept.

32. The method of claim 29 wherein the anti-Tumor Necrosis Factor-α antibody is infliximab.

33. The method of claim 29 wherein the anti-Tumor Necrosis Factor-α antibody is adalimumab.

34. The method of claim 29 wherein the anti-Tumor Necrosis Factor-α antibody is any humanized monoclonal antibody specific for Tumor Necrosis Factor-α.

35. The method of claim 29 wherein the anti-Tumor Necrosis Factor-α antibody is any molecule that contains extracellular portions of a human Tumor Necrosis Factor-α receptor.
